(19) United States
(12) Patent Application Publication
(10) Pub. No.: US 2025/0120974 A1
(43) Pub. Date: Apr. 17, 2025

(54) PDE9 INHIBITORS FOR TREATING SICKLE CELL DISEASE

(71) Applicant: Cardurion Pharmaceuticals, Inc., Burlington, MA (US)

(72) Inventors: Edward George CALAMAI, Boston, MA (US); Deborah Lynn Leithead DOBBINS, Boston, MA (US); Michael Paul DEHART, Boston, MA (US); James MCARTHUR, Arlington, MA (US); Shi Yin FOO, Boston, MA (US)

(21) Appl. No.: 18/989,150

(22) Filed: Dec. 20, 2024

Related U.S. Application Data
(63) Continuation of application No. 17/186,442, filed on Feb. 26, 2021, now Pat. No. 12,213,975.
Application No. 17/186,442 is a continuation of application No. PCT/US2019/048898, filed on Aug. 29, 2019.
(60) Provisional application No. 62/725,725, filed on Aug. 31, 2018.

(51) Int. Cl. A61K 31/506; A61K 9/00; A61K 9/20; A61K 9/28; A61P 7/06

(52) U.S. Cl. CPC A61K 31/506; A61K 9/0053; A61K 9/2009; A61K 9/2013; A61K 9/2054; A61K 9/2059; A61K 9/284; A61P 7/06

(57) ABSTRACT

The present disclosure relates to PDE9 inhibitors, pharmaceutical compositions comprising the PDE9 inhibitors, and methods of using the PDE9 pharmaceutical compositions for the treatment of sickle cell disease (SCD).

### CROSS-REFERENCE TO RELATED APPLICATIONS

This application is a continuation of U.S. application Ser. No. 17/186,442, filed Feb. 26, 2021, which is a continuation of International Application No. PCT/US2019/048898, filed Aug. 29, 2019, which claims the benefit of U.S. Provisional Application No. 62/725,725, filed Aug. 31, 2018, which is incorporated herein by reference in its entirety.

### FIELD OF THE DISCLOSURE

The present disclosure relates to methods of making and using pharmaceutical compositions comprising cyclic guanylate monophosphate (cGMP)-specific phosphodiesterase type 9 inhibitors (hereinafter referred to as PDE9 inhibitors).

### BACKGROUND

Sickle Cell Disease (SCD, also called sickle cell anemia (SCA)) is a genetic disorder leading to vaso-occlusive processes responsible for much of the mortality in SCD patients. SCD disease results from a point mutation in the hemoglobin (HBB) gene producing abnormal sickle hemoglobin (HbS or HbSS), which polymerizes and creates rigid and sticky sickled red blood cells. Sickled red blood cells result in chronic inflammation, elevated cell adhesion, oxidative stress, and endothelial dysfunction culminating in vaso-occlusive processes.

There is to date no cure for SCD. Treatment options include blood transfusion and treatment with the anti-cancer agent hydroxyurea. Blood transfusions correct anemia by increasing the number of normal, non-sickled red blood cells in circulation. Regular transfusion therapy can help prevent recurring strokes in children at high risk. Hydroxyurea (HU) has been approved for the treatment of SCD and shown to reduce the frequency of painful crisis and hospitalization. Unfortunately, HU is often poorly tolerated and its widespread use is limited by concerns about its potential impact on fertility and reproduction; challenges achieving and maintaining an efficacious dose due to its hematologic toxicities; and requirements for monthly monitoring (Heeney et al., Pediatr Clin North Am., 2008, 55 (2): 483). In fact, it is estimated that only 1 out of 4 adult patients, and possibly even fewer, are treated with this drug (Stettler et al., JAMA, 2015, 313:1671). In addition, many patients are dosed with sub-efficacious doses of HU due to these challenges. Thus, novel, safe, and effective treatments that can be safely employed globally to prevent the morbid complications of SCD in patients of all ages are urgently needed.

There remains a need for treating SCD.

### SUMMARY OF THE DISCLOSURE

The present disclosure provides methods of making and using Compound 1 and/or pharmaceutical compositions comprising Compound 1 or a pharmaceutically acceptable salt or polymorph thereof, to treat sickle cell disease.

In one aspect described herein, an oral pharmaceutical composition comprises: about 100 mg to about 300 mg of 6-[(3S,4S)-4-methyl-1-(pyrimidin-2-ylmethyl) pyrrolidin-3-yl]-3-tetrahydropyran-4-yl-7H-imidazo[1,5-a]pyrazin- 8-one (Compound 1), or a pharmaceutically acceptable salt, solvate, or polymorph thereof; a filler selected from about 4% to about 6% by weight of pre-gelatinized starch and/or from about 15% to about 50% microcrystalline cellulose; and a processing aid selected from about 1% to about 2.5% by weight of colloidal silicon dioxide and/or from about 0.5% to about 1.5% by weight of magnesium stearate, the pharmaceutical composition is in the form of a solid tablet suitable for administration to a patient. In some embodiments, the composition has a friability of no more than about 0.3% weight loss and a has a disintegration time of less than about 15 minutes, as determined by USP friability and USP disintegration tests. In some embodiments, the composition has at least one of a friability of no more than about 0.3% weight loss or a disintegration time of less than about 15 minutes, as determined by USP friability and USP disintegration tests. In some embodiments, the pharmaceutical composition above further comprises hydroxypropyl cellulose (HPC). In some embodiments, the hydroxypropyl cellulose (HPC) is present in an amount from about 0.4% to about 0.5% by weight. In some embodiments, the hydroxypropyl cellulose (HPC) is present in an amount of about 0.5% by weight. In some embodiments, the composition has a hardness of about 10 kPa and/or a thickness of about 4.50 to about 4.80 mm. In some embodiments, comprises about 100 mg, 200 mg, or about 300 mg of Compound 1, or a pharmaceutically acceptable salt, solvate, or polymorph thereof. In some embodiments, comprises about 300 mg of Compound 1, or a pharmaceutically acceptable salt, solvate, or polymorph thereof. In some embodiments, comprises about 5% by weight of pre-gelatinized starch. In some embodiments, comprises about 2% by weight of colloidal silicon dioxide. In some embodiments1, comprises about 1% by weight of magnesium stearate. In some embodiments, the composition comprises pre-gelatinized starch, colloidal silicon dioxide, and magnesium stearate at a weight ratio of 5:2:1. In some embodiments, further comprises a coating selected from an enteric coating or Opadry® II white film coating. In some embodiments, the coating is about 2.5% by weight of the total tablet.

In another aspect described herein, an oral pharmaceutical composition comprises: from about 100 mg to about 300 mg of 6-[(3S,4S)-4-methyl-1-(pyrimidin-2-ylmethyl)pyrrolidin-3-yl]-3-tetrahydropyran-4-yl-7H-imidazo[1,5-a]pyrazin-8-one (Compound 1), or a pharmaceutically acceptable salt, solvate, or polymorph thereof; about 5% by weight of pre-gelatinized starch; about 20% by weight of microcrystalline cellulose; about 2% by weight of colloidal silicon dioxide; and about 1% by weight of magnesium stearate, the pharmaceutical composition is in the form of a solid tablet. In some embodiments, the composition has a friability of no more than about 0.3% weight loss and/or a disintegration time of less than about 15 minutes, as determined by USP friability and USP disintegration tests. In some embodiments, further comprises about 0.5% by weight of hydroxypropyl cellulose (HPC). An oral pharmaceutical composition comprises 6-[(3S,4S)-4-methyl-1-(pyrimidin-2-ylmethyl)pyrrolidin-3-yl]-3-tetrahydropyran-4-yl-7H-imidazo[1,5-a]pyrazin-8-one (Compound 1), or a pharmaceutically acceptable salt, solvate, or polymorph thereof; the pharmaceutical composition is in the form of a solid tablet. In some embodiments, further comprises at least a filler. In some embodiments, the filler is microcrystalline cellulose or pre-gelatinized starch. In some embodiments, the composition comprises about 4% to about 6% by weight of pre-gelatinized starch. In some embodiments, the composition comprises about 15 mg to about 25 mg of pre-gelatinized starch per tablet. In some embodiments, the composition further comprises at least a processing aid. In some embodiments, the processing aid is colloidal silicon dioxide and/or magnesium stearate. In some embodiments, the composition comprises about 1% to about 2.5% by weight of colloidal silicon dioxide and/or about 0.5% to about 1.5% by weight of magnesium stearate. In some embodiments, the composition comprises about 6 mg to about 8 mg colloidal silicon dioxide and/or about 2 mg to about 4 mg of magnesium stearate per tablet. In some embodiments, comprises microcrystalline cellulose, pre-gelatinized starch, colloidal silicon dioxide, and magnesium stearate. In some embodiments, further comprises a coating selected from an enteric coating or Opadry® II white film coating. In some embodiments, the coating is about 2.5% by weight of the tablet. In some embodiments, the composition comprises about 20 mg to about 40 mg of the coating per tablet. In some embodiments, the composition comprises pre-gelatinized starch, colloidal silicon dioxide, and magnesium stearate at a weight ratio of 5:2:1. In some embodiments, Compound 1, or a pharmaceutically acceptable salt or polymorph thereof, is present in an amount of about 30 mg, 50 mg, 100 mg, 150 mg, 200 mg, 250 mg, 300 mg, or 350 mg. In some embodiments, Compound 1, or a pharmaceutically acceptable salt or polymorph thereof, is present in the composition in an amount from about 50% to about 80% or from about 60% to about 75% by weight of the solid tablet. In some embodiments, Compound 1, or a pharmaceutically acceptable salt or polymorph thereof, is present in the composition in an amount 65%, about 68%, about 70%, about 72%, or about 75% by weight of the solid tablet. In some embodiments, the composition has a friability and/or a disintegration time. In some embodiments, the composition has a friability of no more than 0.3% weight loss and/or a disintegration time of less than about 15 minutes, friability and disintegration time are determined by USP testing. In some embodiments, the composing further comprises 0.4% to about 0.5% by weight of hydroxypropyl cellulose.

In another aspect described herein, a method for treating sickle cell disease in a subject in need, comprises administering any of the pharmaceutical compositions above. In some embodiments, the pharmaceutical composition is taken with food. In some embodiments, the pharmaceutical composition is administered once per day, twice per day, or three times per day. In some embodiments, the pharmaceutical composition is administered once per day. In some embodiments, the pharmaceutical composition is administered for at least 4 weeks, 12 weeks, 16 weeks, or 24 weeks. In some embodiments, the method further comprises administering hydroxyurea (HU). In some embodiments, the method comprises administering to the subject about 0.3 mg/kg to about 6.0 mg/kg or from about 0.3 mg/kg to about 1.0 mg/kg of subjects body mass per day or per dose of Compound 1, or a pharmaceutically acceptable salt or polymorph thereof.

### BRIEF DESCRIPTION OF THE DRAWINGS

FIG. 1 shows Compound 1 reduces myeloid and neutrophil inflammatory markers in the lungs of Townes mice.

FIG. 2 shows Compound 1 reduces adhesion of SCD patient neutrophils to endothelial cell lined microfluidic chamber in vitro.

FIG. 3 shows Compound 1 reduces expression of CD11a, CD11b and CD18 integrins on SCD patient neutrophils.

FIG. 4 shows a chart of comparison between Compound 1 and hydroxyurea showing the superior efficacy of Compound 1.

FIG. 5 shows the outcome of studies in the Townes SCD Model comparing Compound 1 (30 mg/kg).

FIG. 6 shows the outcome of studies in the Townes SCD Model comparing Compound 1 (30 mg/kg).

FIG. 7 illustrates a clinical study design for Compound 1.

FIG. 8 depicts, without limitation, a representative sampling of screenshots for use in a mobile device running software designed to track human impact of a pharmaceutical.

### DETAILED DESCRIPTION

Phosphodiesterases (PDEs) are a family of enzymes degrading cyclic nucleotides and thereby regulating the cellular levels of second messengers throughout the entire body. PDEs represent attractive drug targets, as proven by a number of compounds that have been introduced to clinical testing and the market, respectively. PDEs are encoded by 21 genes that are functionally separated into 11 families differing with respect to kinetic properties, substrate selectivity, expression, localization pattern, activation, regulation factors and inhibitor sensitivity. The function of PDEs is the degradation of the cyclic nucleotide monophosphates cyclic Adenosine Monophosphate (cAMP) and/or Guanosine Monophosphate (cGMP), which are important intracellular mediators involved in numerous vital processes including the control of neurotransmission and smooth muscle contraction and relaxation.

PDE9 is cGMP specific (Kₘ CAMP is >1000× for cGMP) and is hypothesized to be a key player in regulating cGMP levels as it has the lowest Kₘ among the PDEs for this nucleotide. PDE9 is expressed throughout the brain at low levels with the potential for regulating basal cGMP.

In the periphery, PDE9 expression is highest in prostate, intestine, kidney and haematopoietic cells, enabling therapeutic potential in various non-CNS indications.

In the present disclosure, pharmaceutical compositions comprising PDE9 inhibitors are designed for treatment for Sickle Cell Disease (SCD).

### Compounds of the Disclosure

In the context of the present disclosure, a compound is considered to be a PDE9 inhibitor if the amount required to reach the 50% inhibition level PDE9 is 10 micromolar or less, preferably less than 9 micromolar, such as 8 micromolar or less, such as 7 micromolar or less, such as 6 micromolar or less, such as 5 micromolar or less, such as 4 micromolar or less, such as 3 micromolar or less, more preferably 2 micromolar or less, such as 1 micromolar or less, in particular 500 nM or less. In preferred embodiments the required amount of PDE9inhibitor required to reach the IC₅₀ level of PDE9 is 400nM or less, such as 300 nM or less, 200 nM or less, 100 nM or less, or even 80 nM or less, such as 50 nM or less, for example 25 nM or less.

Throughout this application the notations IC₅₀ and IC50 are used interchangeably.

In some embodiments, the PDE9 inhibitor of the present disclosure has low or no blood brain barrier penetration. For example, the ratio of the concentration of a PDE9 inhibitor of the present disclosure in the brain to the concentration of it in the plasma (brain/plasma ratio) may be less than about 0.50, about 0.40, about 0.30, about 0.20, about 0.10, about 0.05, about 0.04, about 0.03, about 0.02, or about 0.01. In some embodiments, the brain/plasma ration is measured 30 min or 120 min after administration of the PDE9 inhibitor.

In some embodiments, the PDE9 inhibitor may be any imidazo pyrazinone PDE9 inhibitor disclosed in WO 2013/053690 and/or any imidazo triazinone PDE9 inhibitor disclosed in WO 2013/110768, the contents of each of which are incorporated herein by reference in their entirety.

In some embodiments, the PDE9 inhibitor is Compound 1 or a pharmaceutically acceptable salt, cocrystal, solvate, or polymorph thereof. A racemate form of Compound 1 and an anhydrous form of Compound 1 have been described in WO 2013/053690 and WO 2017/005786. In some embodiments, the PDE9 inhibitor is 6-[(3S,4S)-4-methyl-1-(pyrimidin-2-ylmethyl)pyrrolidin-3-yl]-3-tetrahydropyran-4-yl-7H-imidazo[1,5-a]pyrazin-8-one (Compound 1), or a pharmaceutically acceptable salt or polymorph thereof. In some

Compound 1 has the following structure:

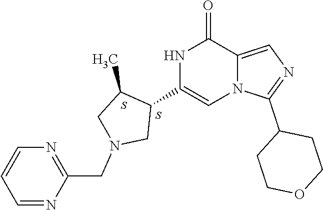

6-[(3S,4S)-4-methyl-1-(pyrimidin-2-ylmethyl)pyrrolidin-3-yl]-3-tetrahydropyran-4-yl-7H-imidazo[1,5-a]pyrazin-8-one; Formula C₂₁H₂₆N₆O₂; calculated molecular weight about 394 g/mol. In some embodiments, Compound 1 is enantiopure or substantially enantiopure.

### Pharmaceutical Compositions

The present disclosure further provides a pharmaceutical composition comprising a therapeutically effective amount of any of the PDE9 inhibitors and a pharmaceutically acceptable carrier or diluent. In some embodiments, the present disclosure provides a pharmaceutical composition comprising a therapeutically effective amount of Compound 1, or a pharmaceutically acceptable salt or polymorph thereof, and a pharmaceutically acceptable carrier or diluent or excipient.

### Pharmaceutically Acceptable Salts

The present disclosure also comprises salts of the PDE9 inhibitors, typically, pharmaceutically acceptable salts. Such salts include pharmaceutically acceptable acid addition salts. Acid addition salts include salts of inorganic acids as well as organic acids.

Representative examples of suitable inorganic acids include hydrochloric, hydrobromic, hydroiodic, phosphoric, sulfuric, sulfamic, nitric acids and the like. Representative examples of suitable organic acids include formic, acetic, trichloroacetic, propionic, benzoic, cinnamic, citric, fumaric, glycolic, itaconic, lactic, methanesulfonic, maleic, malic, malonic, mandelic, oxalic, picric, pyruvic, salicylic, succinic, methane sulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, theophylline acetic acids, as well as the 8-halotheophyllines, for example 8-bromotheophylline and the like. Further examples of pharmaceutically acceptable inorganic or organic acid addition salts include the pharmaceutically acceptable salts listed in Berge, S. M. et al., J. Pharm. Sci. 1977, 66, 2, the contents of which are hereby incorporated by reference.

Furthermore, the compounds of this disclosure may exist in unsolvated as well as in solvated forms with pharmaceutically acceptable solvents such as water, ethanol, and the like. In general, the solvated forms are considered equivalent to the unsolvated forms for the purposes of this disclosure.

In some embodiments, the pharmaceutical composition comprises Compound 1 as the solvated, unsolvated, or crystalline form. In some embodiments, Compound 1 is present as the unsolvated form. In some embodiments, Compound 1 is present as the present as the crystalline form. In some embodiments, Compound 1 is present as a monohydrate crystalline form.

### Formulations

The compounds of the disclosure may be administered alone or in combination with pharmaceutically acceptable carriers, diluents or excipients, in either single or multiple doses. The pharmaceutical compositions according to the disclosure may be formulated with pharmaceutically acceptable carriers or diluents as well as any other known adjuvants and excipients in accordance with conventional techniques such as those disclosed in Remington: The Science and Practice of Pharmacy, 22nd Edition, Gennaro, Ed., Mack Publishing Co., Easton, PA, 2013.

The pharmaceutical compositions may be specifically formulated for administration by any suitable route, such as oral, rectal, nasal, pulmonary, topical (including buccal and sublingual), transdermal, intracisternal, intraperitoneal, vaginal, and parenteral (including subcutaneous, intramuscular, intrathecal, intravenous, and intradermal) routes. It will be appreciated that the route will depend on the general health and age of the subject to be treated, the nature of the condition to be treated, and the active ingredient. In some embodiments, the pharmaceutical composition is formulated for oral administration to a subject. In some embodiments, the pharmaceutical composition is formulated as a tablet or pill. In some embodiments, the pharmaceutical composition is formulated as a solid tablet suitable for oral administration to a subject.

Pharmaceutical compositions for oral administration include solid dosage forms such as capsules, tablets, dragees, pills, lozenges, powders, and granules. Where appropriate, the compositions may be prepared with coatings, such as enteric coatings or they may be formulated so as to provide controlled release of the active ingredient such as sustained or prolonged release according to methods well known in the art. Liquid dosage forms for oral administration include solutions, emulsions, suspensions, syrups, and elixirs, either manufactured as such, or as a solid form for reconstitution prior to use.

Pharmaceutical compositions for parenteral administration include sterile aqueous and non-aqueous injectable solutions, dispersions, suspensions, or emulsions as well as sterile powders to be reconstituted in sterile injectable solutions or dispersions prior to use. Other suitable administration forms include, but are not limited to, suppositories, sprays, ointments, creams, gels, inhalants, dermal patches, and implants.

The present disclosure also provides a process for making a pharmaceutical composition comprising admixing a therapeutically effective amount of a compound of the present disclosure and at least one pharmaceutically acceptable carrier or diluent.

The compounds of this disclosure are generally utilized as the free substance or as a pharmaceutically acceptable salt thereof. Such salts are prepared in a conventional manner by treating a solution or suspension of a compound of the present disclosure with a pharmaceutically acceptable acid. Representative examples of suitable organic and inorganic acids are described above.

For parenteral administration, solutions of the compounds of the present disclosure in sterile aqueous solution, aqueous propylene glycol, aqueous vitamin E or sesame or peanut oil may be employed. Such aqueous solutions should be suitably buffered if necessary and the liquid diluent first rendered isotonic with sufficient saline or glucose. The aqueous solutions are particularly suitable for intravenous, intramuscular, subcutaneous and intraperitoneal administration. The compounds of the present disclosure may be readily incorporated into known sterile aqueous media using standard techniques known to those skilled in the art.

Suitable pharmaceutical carriers include inert solid diluents or fillers, sterile aqueous solutions and various organic solvents. Examples of solid carriers include lactose, terra alba, sucrose, cyclodextrin, talc, gelatin, agar, pectin, acacia, magnesium stearate, stearic acid and lower alkyl ethers of cellulose. Examples of liquid carriers include, but are not limited to, syrup, peanut oil, olive oil, phospholipids, fatty acids, fatty acid amines, polyoxyethylene and water. Similarly, the carrier or diluent may include any sustained release material known in the art, such as glyceryl monostearate or glyceryl distearate, alone or mixed with a wax. The pharmaceutical compositions formed by combining the compounds of the present disclosure and a pharmaceutically acceptable carrier are then readily administered in a variety of dosage forms suitable for the disclosed routes of administration. The formulations may conveniently be presented in unit dosage form by methods known in the art of pharmacy.

Pharmaceutical compositions of the present disclosure suitable for oral administration may be presented as discrete units such as capsules or tablets, each containing a predetermined amount of the active ingredient, and optionally a suitable excipient. Furthermore, the orally available formulations may be in the form of a powder or granules, a solution or suspension in an aqueous or non-aqueous liquid, or an oil-in-water or water-in-oil liquid emulsion.

If a solid carrier is used for oral administration, the preparation may be tabletted, placed in a hard gelatine capsule in powder or pellet form or it may be in the form of a troche or lozenge. The amount of solid carrier will vary widely but will range from about 25 mg to about 1 g per dosage unit. In some embodiments, the solid carrier will be about 10 mg to about 150 mg per dosage unit. In some embodiments, the solid carrier will be about 10 mg to about 20 mg, about 10 mg to about 30 mg, about 10 mg to about 40 mg, about 10 mg to about 50 mg, about 10 mg to about 60 mg, about 10 mg to about 70 mg, about 10 mg to about 80 mg, about 10 mg to about 90 mg, about 10 mg to about 100 mg, about 10 mg to about 125 mg, about 10 mg to about 150 mg, about 20 mg to about 30 mg, about 20 mg to about 40 mg, about 20 mg to about 50 mg, about 20 mg to about 60 mg, about 20 mg to about 70 mg, about 20 mg to about 80 mg, about 20 mg to about 90 mg, about 20 mg to about 100 mg, about 20 mg to about 125 mg, about 20 mg to about 150 mg, about 30 mg to about 40 mg, about 30 mg to about 50 mg, about 30 mg to about 60 mg, about 30 mg to about 70 mg, about 30 mg to about 80 mg, about 30 mg to about 90 mg, about 30 mg to about 100 mg, about 30 mg to about 125 mg, about 30 mg to about 150 mg, about 40 mg to about 50 mg, about 40 mg to about 60 mg, about 40 mg to about 70 mg, about 40 mg to about 80 mg, about 40 mg to about 90 mg, about 40 mg to about 100 mg, about 40 mg to about 125 mg, about 40 mg to about 150 mg, about 50 mg to about 60 mg, about 50 mg to about 70 mg, about 50 mg to about 80 mg, about 50 mg to about 90 mg, about 50 mg to about 100 mg, about 50 mg to about 125 mg, about 50 mg to about 150 mg, about 60 mg to about 70 mg, about 60 mg to about 80 mg, about 60 mg to about 90 mg, about 60 mg to about 100 mg, about 60 mg to about 125 mg, about 60 mg to about 150 mg, about 70 mg to about 80 mg, about 70 mg to about 90 mg, about 70 mg to about 100 mg, about 70 mg to about 125 mg, about 70 mg to about 150 mg, about 80 mg to about 90 mg, about 80 mg to about 100 mg, about 80 mg to about 125 mg, about 80 mg to about 150 mg, about 90 mg to about 100 mg, about 90 mg to about 125 mg, about 90 mg to about 150 mg, about 100 mg to about 125 mg, about 100 mg to about 150 mg, or about 125 mg to about 150 mg. In some embodiments, the solid carrier will be about 10 mg, about 20 mg, about 30 mg, about 40 mg, about 50 mg, about 60 mg, about 70 mg, about 80 mg, about 90 mg, about 100 mg, about 125 mg, or about 150 mg. In some embodiments, the solid carrier will be at least about 10 mg, about 20 mg, about 30 mg, about 40 mg, about 50 mg, about 60 mg, about 70 mg, about 80 mg, about 90 mg, about 100 mg, or about 125 mg. In some embodiments, the solid carrier will be at most about 20 mg, about 30 mg, about 40 mg, about 50 mg, about 60 mg, about 70 mg, about 80 mg, about 90 mg, about 100 mg, about 125 mg, or about 150 mg per dosage unit. In some embodiments, the solid carrier will be about 150 mg to about 1,000 mg per dosage unit. In some embodiments, the solid carrier will be about 150 mg to about 175 mg, about 150 mg to about 200 mg, about 150 mg to about 300 mg, about 150 mg to about 400 mg, about 150 mg to about 500 mg, about 150 mg to about 600 mg, about 150 mg to about 700 mg, about 150 mg to about 800 mg, about 150 mg to about 900 mg, about 150 mg to about 1,000 mg, about 175 mg to about 200 mg, about 175 mg to about 300 mg, about 175 mg to about 400 mg, about 175 mg to about 500 mg, about 175 mg to about 600 mg, about 175 mg to about 700 mg, about 175 mg to about 800 mg, about 175 mg to about 900 mg, about 175 mg to about 1,000 mg, about 200 mg to about 300 mg, about 200 mg to about 400 mg, about 200 mg to about 500 mg, about 200 mg to about 600 mg, about 200 mg to about 700 mg, about 200 mg to about 800 mg, about 200 mg to about 900 mg, about 200 mg to about 1,000 mg, about 300 mg to about 400 mg, about 300 mg to about 500 mg, about 300 mg to about 600 mg, about 300 mg to about 700 mg, about 300 mg to about 800 mg, about 300 mg to about 900 mg, about 300 mg to about 1,000 mg, about 400 mg to about 500 mg, about 400 mg to about 600 mg, about 400 mg to about 700 mg, about 400 mg to about 800 mg, about 400 mg to about 900 mg, about 400 mg to about 1,000 mg, about 500 mg to about 600 mg, about 500 mg to about 700 mg, about 500 mg to about 800 mg, about 500 mg to about 900 mg, about 500 mg to about 1,000 mg, about 600 mg to about 700 mg, about 600 mg to about 800 mg, about 600 mg to about 900 mg, about 600 mg to about 1,000 mg, about 700 mg to about 800 mg, about 700 mg to about 900 mg, about 700 mg to about 1,000 mg, about 800 mg to about 900 mg, about 800 mg to about 1,000 mg, or about 900 mg to about 1,000 mg. In some embodiments, the solid carrier will be about 150 mg, about 175 mg, about 200 mg, about 300 mg, about 400 mg, about 500 mg, about 600 mg, about 700 mg, about 800 mg, about 900 mg, or about 1,000 mg. In some embodiments, the solid carrier will be at least about 150 mg, about 175 mg, about 200 mg, about 300 mg, about 400 mg, about 500 mg, about 600 mg, about 700 mg, about 800 mg, or about 900 mg. In some embodiments, the solid carrier will be at most about 175 mg, about 200 mg, about 300 mg, about 400 mg, about 500 mg, about 600 mg, about 700 mg, about 800 mg, about 900 mg, or about 1,000 mg per dosage unit.

In some embodiments, the solid carrier will be about 10 mg, 15 mg, 20 mg, 25mg, 30 mg, 35 mg, 40 mg, 45 mg, 50 mg, 55 mg, 60 mg, 65 mg, 70 mg, 75 mg, 80 mg, 85 mg, 90 mg, 95 mg, or about 1 g per dosage unit. If a liquid carrier is used, the preparation may be in the form of a syrup, emulsion, soft gelatine capsule or sterile injectable liquid such as an aqueous or non-aqueous liquid suspension or solution

In some embodiments, the solid carrier will be about 1 g to about 2 g per dosage unit. In some embodiments, the solid carrier will be about 1 g to about 1.1 g, about 1 g to about 1.2 g, about 1 g to about 1.3 g, about 1 g to about 1.4 g, about 1 g to about 1.5 g, about 1 g to about 1.6 g, about 1 g to about 1.7 g, about 1 g to about 1.8 g, about 1 g to about 1.9 g, about 1 g to about 2 g, about 1.1 g to about 1.2 g, about 1.1 g to about 1.3 g, about 1.1 g to about 1.4 g, about 1.1 g to about 1.5 g, about 1.1 g to about 1.6 g, about 1.1 g to about 1.7 g, about 1.1 g to about 1.8 g, about 1.1 g to about 1.9 g, about 1.1 g to about 2 g, about 1.2 g to about 1.3 g, about 1.2 g to about 1.4 g, about 1.2 g to about 1.5 g, about 1.2 g to about 1.6 g, about 1.2 g to about 1.7 g, about 1.2 g to about 1.8 g, about 1.2 g to about 1.9 g, about 1.2 g to about 2 g, about 1.3 g to about 1.4 g, about 1.3 g to about 1.5 g, about 1.3 g to about 1.6 g, about 1.3 g to about 1.7 g, about 1.3 g to about 1.8 g, about 1.3 g to about 1.9 g, about 1.3 g to about 2 g, about 1.4 g to about 1.5 g, about 1.4 g to about 1.6 g, about 1.4 g to about 1.7 g, about 1.4 g to about 1.8 g, about 1.4 g to about 1.9 g, about 1.4 g to about 2 g, about 1.5 g to about 1.6 g, about 1.5 g to about 1.7 g, about 1.5 g to about 1.8 g, about 1.5 g to about 1.9 g, about 1.5 g to about 2 g, about 1.6 g to about 1.7 g, about 1.6 g to about 1.8 g, about 1.6 g to about 1.9 g, about 1.6 g to about 2 g, about 1.7 g to about 1.8 g, about 1.7 g to about 1.9 g, about 1.7 g to about 2 g, about 1.8 g to about 1.9 g, about 1.8 g to about 2 g, or about 1.9 g to about 2 g. In some embodiments, the solid carrier will be about 1 g, about 1.1 g, about 1.2 g, about 1.3 g, about 1.4 g, about 1.5 g, about 1.6 g, about 1.7 g, about 1.8 g, about 1.9 g, or about 2 g. In some embodiments, the solid carrier will be at least about 1 g, about 1.1 g, about 1.2 g, about 1.3 g, about 1.4 g, about 1.5 g, about 1.6 g, about 1.7 g, about 1.8 g, or about 1.9 g. In some embodiments, the solid carrier will be at most about 1.1 g, about 1.2 g, about 1.3 g, about 1.4 g, about 1.5 g, about 1.6 g, about 1.7 g, about 1.8 g, about 1.9 g, or about 2 g per dosage unit.

The pharmaceutical compositions of the disclosure may be prepared by conventional methods in the art. For example, tablets may be prepared by mixing the active ingredient with ordinary adjuvants and/or diluents and subsequently compressing the mixture in a conventional tabletting machine prepare tablets. Examples of adjuvants or diluents comprise: corn starch, potato starch, talcum, magnesium stearate, gelatin, lactose, gums, and the like. Any other adjuvants or additives usually used for such purposes such as colorings, flavorings, preservatives etc. may be used provided that they are compatible with the active ingredients.

The pharmaceutical composition comprises PDE9 inhibitor Compound 1. The pharmaceutical composition comprises at least 10%, 20%, 30%, 40%, 50%, 60%, 70%, 80%, or 90% by weight of PDE9 inhibitors of the present disclosure. The pharmaceutical composition comprises at least about 1% to about 90% by weight of PDE9 inhibitors of the present disclosure. The pharmaceutical compositions comprises at least about 1% to about 10%, about 1% to about 20%, about 1% to about 30%, about 1% to about 40%, about 1% to about 50%, about 1% to about 60%, about 1% to about 70%, about 1% to about 80%, about 1% to about 90%, about 10% to about 20%, about 10% to about 30%, about 10% to about 40%, about 10% to about 50%, about 10% to about 60%, about 10% to about 70%, about 10% to about 80%, about 10% to about 90%, about 20% to about 30%, about 20% to about 40%, about 20% to about 50%, about 20% to about 60%, about 20% to about 70%, about 20% to about 80%, about 20% to about 90%, about 30% to about 40%, about 30% to about 50%, about 30% to about 60%, about 30% to about 70%, about 30% to about 80%, about 30% to about 90%, about 40% to about 50%, about 40% to about 60%, about 40% to about 70%, about 40% to about 80%, about 40% to about 90%, about 50% to about 60%, about 50% to about 70%, about 50% to about 80%, about 50% to about 90%, about 60% to about 70%, about 60% to about 80%, about 60% to about 90%, about 70% to about 80%, about 70% to about 90%, or about 80% to about 90%. The pharmaceutical compositions comprise at least about 1%, about 10%, about 20%, about 30%, about 40%, about 50%, about 60%, about 70%, about 80%, or about 90%. The pharmaceutical compositions comprises at least at least about 1%, about 10%, about 20%, about 30%, about 40%, about 50%, about 60%, about 70%, or about 80%. The pharmaceutical compositions comprises at least at most about 10%, about 20%, about 30%, about 40%, about 50%, about 60%, about 70%, about 80%, or about 90% by weight of PDE9 inhibitors of the present disclosure. The pharmaceutical composition comprises at least about 90% to about 99.9% by weight of PDE9 inhibitors of the present disclosure. The pharmaceutical composition comprises at least about 90% to about 91%, about 90% to about 92%, about 90% to about 93%, about 90% to about 94%, about 90% to about 95%, about 90% to about 96%, about 90% to about 97%, about 90% to about 98% about 90% to about 99%, about 90% to about 99.9%, about 91% to about 92%, about 91% to about 93%, about 91% to about 94%, about 91% to about 95%, about 91% to about 96%, about 91% to about 97%, about 91% to about 98%, about 91% to about 99%, about 91% to about 99.9%, about 92% to about 93%, about 92% to about 94%, about 92% to about 95%, about 92% to about 96%, about 92% to about 97%, about 92% to about 98%, about 92% to about 99%, about 92% to about 99.9%, about 93% to about 94%, about 93% to about 95%, about 93% to about 96%, about 93% to about 97%, about 93% to about 98%, about 93% to about 99%, about 93% to about 99.9%, about 94% to about 95%, about 94% to about 96%, about 94% to about 97%, about 94% to about 98%, about 94% to about 99%, about 94% to about 99.9%, about 95% to about 96%, about 95% to about 97%, about 95% to about 98%, about 95% to about 99%, about 95% to about 99.9%, about 96% to about 97%, about 96% to about 98%, about 96% to about 99%, about 96% to about 99.9%, about 97% to about 98%, about 97% to about 99%, about 97% to about 99.9%, about 98% to about 99%, about 98% to about 99.9%, or about 99% to about 99.9%. The pharmaceutical composition comprises at least about 90%, about 91%, about 92%, about 93%, about 94%, about 95%, about 96%, about 97%, about 98%, about 99%, or about 99.9%. The pharmaceutical compositions comprises at least at least about 90%, about 91%, about 92%, about 93%, about 94%, about 95%, about 96%, about 97%, about 98%, or about 99%. The pharmaceutical composition comprises at least at most about 91%, about 92%, about 93%, about 94%, about 95%, about 96%, about 97%, about 98%, about 99%, or about 99.9% by weight of PDE9 inhibitors of the present disclosure. The pharmaceutical composition comprise at least 90%, 91%, 92%, 93%, 94%, 95%, 96%, 97%, 98%, or 99% by weight of PDE9 inhibitors of the present disclosure.

In some embodiments, Compound 1 or a pharmaceutically acceptable salt or polymorph thereof is formulated as a pharmaceutical composition for oral administration. For example, it may be in a solid tablet form. The composition for oral administration comprises at least a filler and/or a processing aid. The processing aid may be a glidant or a lubricant. The composition for oral administration may also comprise a coating. In some embodiments, the composition for oral administration comprises microcrystalline cellulose and/or pre-gelatinized starch as fillers. In some embodiments, the composition for oral administration comprises colloidal silicon dioxide and/or magnesium stearate as processing aids. In some embodiments, the composition for oral administration comprises Opadry® II white film coating. Opadry® II is a high productivity, water soluble, pH independent complete dry powder film coating system containing polymer, plasticizer and pigment which allows for immediate disintegration for fast, active release. In some embodiments, the composition for oral administration comprises purified water, which is removed during processing.

In some embodiments, the tablet comprises a coating between about 5% to about 20% by weight of the total weight of the tablet. In some embodiments, the tablet comprises a coating between about 0.5% to about 10%. In some embodiments, the tablet comprises a coating between about 0.5% to about 1%, about 0.5% to about 2%, about 0.5% to about 3%, about 0.5% to about 4%, about 0.5% to about 5%, about 0.5% to about 6%, about 0.5% to about 7%, about 0.5% to about 8%, about 0.5% to about 9%, about 0.5% to about 10%, about 1% to about 2%, about 1% to about 3%, about 1% to about 4%, about 1% to about 5%, about 1% to about 6%, about 1% to about 7%, about 1% to about 8%, about 1% to about 9%, about 1% to about 10%, about 2% to about 3%, about 2% to about 4%, about 2% to about 5%, about 2% to about 6%, about 2% to about 7%, about 2% to about 8%, about 2% to about 9%, about 2% to about 10%, about 3% to about 4%, about 3% to about 5%, about 3% to about 6%, about 3% to about 7%, about 3% to about 8%, about 3% to about 9%, about 3% to about 10%, about 4% to about 5%, about 4% to about 6%, about 4% to about 7%, about 4% to about 8%, about 4% to about 9%, about 4% to about 10%, about 5% to about 6%, about 5% to about 7%, about 5% to about 8%, about 5% to about 9%, about 5% to about 10%, about 6% to about 7%, about 6% to about 8%, about 6% to about 9%, about 6% to about 10%, about 7% to about 8%, about 7% to about 9%, about 7% to about 10%, about 8% to about 9%, about 8% to about 10%, or about 9% to about 10%. In some embodiments, the tablet comprises a coating between about 0.5%, about 1%, about 2%, about 3%, about 4%, about 5%, about 6%, about 7%, about 8%, about 9%, or about 10%. In some embodiments, the tablet comprises a coating between at least about 0.5%, about 1%, about 2%, about 3%, about 4%, about 5%, about 6%, about 7%, about 8%, or about 9%. In some embodiments, the tablet comprises a coating between at most about 1%, about 2%, about 3%, about 4%, about 5%, about 6%, about 7%, about 8%, about 9%, or about 10%. In some embodiments, the tablet comprises a coating between about 0.5% about 5%, about 10%, about 15%, or about 20% by weight of the total weight of the tablet.

In some embodiments, the composition further comprises an enteric coating. An enteric coating is a polymer barrier applied on oral medication that prevents its dissolution or disintegration in the gastric environment. Tablets, mini-tablets, pellets and granules (usually filled into capsule shells) are the most common enteric-coated dosage forms. Most enteric coatings work by presenting a surface that is stable at the intensely acidic pH found in the stomach, but breaks down rapidly at a higher pH (alkaline pH). For example, they will not dissolve in the gastric acids of the stomach (pH ˜3), but they will in the alkaline (pH 7-9) environment present in the small intestine. By preventing the drug from dissolving into the stomach, enteric coating may protect gastric mucosa from the irritating effects of the medication itself. When the drug reaches the neutral or alkaline environment of the intestine, its active ingredients can then dissolve and become available for absorption into the bloodstream. Materials used for enteric coatings include but are not limited to fatty acids, waxes, shellac, plastics, and plant fibers. Conventional materials used are solutions of film resins.

In some embodiments, the pharmaceutical composition comprises a filler. In some embodiments, the filler is pre-geletinized starch. In the embodiment, the pharmaceutical composition comprises pre-gelatinized starch between about 4% to about 6% by weight of the total weight of the pharmaceutical composition. In some embodiments, the pharmaceutical composition comprises pre-gelatinized starch at about 4%, about 5%, or about 6% by weight of the total weight of the pharmaceutical composition.

In some embodiments, the filler is microcrystalline cellulose. In some embodiments, the pharmaceutical composition comprises microcrystalline cellulose at between about 15% to about 50% by weight of the total weight of the pharmaceutical composition. In some embodiments, the pharmaceutical composition comprises microcrystalline cellulose at about 50%, about 40%, about 30%, about 20%, or about 15% by weight of the total weight of the pharmaceutical composition. In some embodiments, the pharmaceutical composition comprises about 20% by weight of microcrystalline cellulose.

In some embodiments, the pharmaceutical composition comprises a processing aid. In some embodiments, the processing aid is colloidal silicon dioxide. In the embodiment, the pharmaceutical composition comprises colloidal silicon dioxide between about 1% to about 2.5% by weight of the total weight of the pharmaceutical composition. In some embodiments, the pharmaceutical composition comprises colloidal silicon dioxide at about 1%, about 1.5%, about 2%, or about 2.5% by weight of the total weight of the pharmaceutical composition.

In some embodiments, the processing aid is magnesium stearate. In the embodiment, the pharmaceutical composition comprises magnesium stearate between about 0.5% to about 1.5% by weight of the total weight of the pharmaceutical composition. In some embodiments, the pharmaceutical composition comprises magnesium stearate at about 0.5%, about 0.8%, about 1.0%, about 1.2%, or about 1.5% by weight of the total weight of the pharmaceutical composition.

In some embodiments, the pharmaceutical composition comprises pre-gelatinized starch, colloidal silicon dioxide, and magnesium stearate at a weight ratio of 5:2:1.

In some embodiments, the pharmaceutical composition comprising Compound 1 or a pharmaceutically acceptable salt or polymorph thereof, is stored at controlled room temperature (20-25° C.).

In some embodiments, the pharmaceutical composition comprising Compound 1 or a pharmaceutically acceptable salt or polymorph thereof, is protected from light.

In some embodiments, the pharmaceutical composition has a friability and/or a disintegration time. Friability, as disclosed herein, is the tendency for a tablet to chip, crumble or break following compression. Friability testing is a laboratory technique to test the durability of tablets. This testing involves repeatedly dropping a sample of tablets over a fixed time, using a rotating wheel with a baffle. Friability is determined by the USP (United States Pharmacopeia) standard test for tablet friability. In some embodiments, the pharmaceutical composition has a friability of no more than about 0.5% weight loss of the tablet. In some embodiments, the pharmaceutical composition has a friability of no more than about 0.4% weight loss of the tablet. In some embodiments, the pharmaceutical composition has a friability of no more than about 0.3% weight loss of the tablet. In some embodiments, the pharmaceutical composition has a friability of no more than about 0.25% weight loss of the tablet.

Disintegration tests are used to test how a drug in pellet form will disintegrate in solution. These tests can be correlated with the in vitro breakdown of powdered compounds for quality control purposes. Disintegration is defined as that state in which no residue of the unit under test remains on the screen of the apparatus or, if a residue remains, it consists of fragments of disintegrated parts of tablets component parts such as insoluble coating of the tablets or of capsule shells, or of any melted fatty substance. This test is most often performed on products that have known absorption problems or known poor solubility. It is also performed on sustained or delayed release products such as enteric coated products. Dissolution testing can be carried out on either capsules or tablets. In some embodiments, the pharmaceutical composition has a dissolution time of no more than 15 minutes.

In some embodiments, the pharmaceutical composition further comprises hydroxypropyl cellulose. Hydroxypropyl cellulose is a derivative of cellulouse with both water and organic solubility. It is often used as a pharmaceutical excipient. Hyrdoxypropyl cellulose can be used as a tablet binder, thickening agent, viscosity-increasing agent, a coating agent, and a film forming agent. In some embodiments, the pharmaceutical composition further comprises from about 1% to about 6% by weight of the table of hydroxypropyl cellulose. In some embodiments, the pharmaceutical composition further comprises from about 4% to about 5% by weight of the table of hydroxypropyl cellulose. In some embodiments, hydroxypropyl cellulose is present at about 4% weight of the tablet. In some embodiments, hydroxypropyl cellulose is present at about 5% weight of the tablet. In some embodiments, hydroxypropyl cellulose is present at about 6% weight of the tablet.

In some embodiments, the pharmaceutical composition has a tablet hardness and/or a tablet thickness. Tablet hardness testing, is a laboratory technique used by the pharmaceutical industry to determine the breaking point and structural integrity of a tablet and find out how it changes under conditions of storage, transportation, packaging and handling before usage. The breaking point of a tablet is based on its shape. In some embodiments, the table has a hardness of about 10 kPa. In some embodiments, the tablet has a thickness of about 4.5-5.0 mm. In some embodiments, the tablet has a thickness of about 4.65-4.85 mm.

In some other embodiments, the composition comprising Compound 1 or a pharmaceutically acceptable salt or polymorph thereof, is suitable for pediatric uses and can be taken by pediatric sickle cell anemia patients.

In some embodiments, the pharmaceutical composition comprising Compound 1 or a pharmaceutically acceptable salt or polymorph thereof, is taken with food. In some embodiments, the pharmaceutical composition, is taken after a meal. In some embodiments, the pharmaceutical composition, is taken without food.

### Dosing

In some embodiments, the oral dosage ranges from about 0.001 to about 100 mg/kg body weight per day. In some embodiments, the oral dosage range is from about 0.01 to about 50 mg/kg body weight per day. In some embodiments, the oral dosage range is from about 0.05 to about 10 mg/kg body weight per day. Oral dosages are usually administered in one or more dosages, typically, one to three dosages per day. In some embodiments, the dose is administered once, twice, or three times a day. The exact dosage will depend upon the frequency and mode of administration, the gender, age, weight, and general health of the subject treated, the nature and severity of the condition treated and any concomitant diseases to be treated and other factors evident to those skilled in the art.

In some embodiments, Compound 1 or a pharmaceutically acceptable salt or polymorph thereof is administered to a subject in need thereof, at a dose of less than 6.0 mg/kg or less than about 4.0 mg/kg per body weight of the subject. In some embodiments, Compound 1 or a pharmaceutically acceptable salt or polymorph thereof, is administered at a dose of from about 0.1 mg/kg to about 6.0 mg/kg per body weight of the subject. For example, Compound 1 or a pharmaceutically acceptable salt or polymorph thereof, is administered at a dose of from about 0.3 to about 3.0 mg/kg, or from about 0.3 to about 1.0 mg/kg per body weight of the subject. The patient may have sickle cell disease. The patient may be an adult (≥18 years old) or a child (<18 years old). In some embodiments, the patient receives Compound 1 or a pharmaceutically acceptable salt or polymorph thereof at a dose of around 0.3 mg/kg, around 0.2 mg/kg, around 0.1 mg/kg, or around 0.05 mg/kg per body weight of the subject. In some embodiments, the patient receives Compound 1 or a pharmaceutically acceptable salt or polymorph thereof, at about 1 mg/kg per body weight of the subject. In some embodiments, the patient receives Compound 1 or a pharmaceutically acceptable salt or polymorph thereof, at about 3 mg/kg per body weight of the subject. In some embodiments, the patient receives Compound 1 or a pharmaceutically acceptable salt or polymorph thereof, at about 6 mg/kg per body weight of the subject.

In some embodiments, the patient receives Compound 1 or a pharmaceutically acceptable salt or polymorph thereof, at about 0.1 mg/kg per body weight of the subject.

In some embodiments, the patient receives Compound 1 or a pharmaceutically acceptable salt or polymorph thereof, at about 0.3 mg/kg per body weight of the subject.

In some embodiments, the patient receives Compound 1 or a pharmaceutically acceptable salt or polymorph thereof, at about 0.5 mg/kg per body weight of the subject.

In some embodiments, the patient receives Compound 1 or a pharmaceutically acceptable salt or polymorph thereof, at about 1 mg/kg per body weight of the subject.

In some embodiments, the patient receives Compound 1 or a pharmaceutically acceptable salt or polymorph thereof, at about 5 mg/kg per body weight of the subject.

In some embodiments, the patient receives Compound 1 or a pharmaceutically acceptable salt or polymorph thereof, at about 10 mg/kg per body weight of the subject.

In some embodiments, Compound 1 or a pharmaceutically acceptable salt or polymorph thereof is administered to a patient in need thereof, at a flat dose of about 20 mg, about 50 mg, about 100 mg, about 200 mg, about 300 mg, about 400, about 500 mg, or about 600 mg per day. In some embodiments, Compound 1 or a pharmaceutically acceptable salt or polymorph thereof is administered to a patient at a dose of about 50 mg, about 100 mg, about 150 mg, about 200 mg, about 250 mg, about 300 mg, or about 350 mg. In some embodiments, Compound 1 or a pharmaceutically acceptable salt or polymorph thereof is administered at a dose of about 50 mg. In some embodiments, Compound 1 or a pharmaceutically acceptable salt or polymorph thereof is administered at a dose of about 100 mg. In some embodiments, Compound 1 or a pharmaceutically acceptable salt or polymorph thereof is administered at a dose of about 150 mg. In some embodiments, Compound 1 or a pharmaceutically acceptable salt or polymorph thereof is administered at a dose of about 200 mg. In some embodiments, Compound 1 or a pharmaceutically acceptable salt or polymorph thereof is administered at a dose of about 250 mg. In some embodiments, Compound 1 or a pharmaceutically acceptable salt or polymorph thereof is administered at a dose of about 300 mg. In some embodiments, Compound 1 or a pharmaceutically acceptable salt or polymorph thereof is administered at a dose of about 350 mg. In some embodiments, Compound 1 or a pharmaceutically acceptable salt or polymorph thereof is administered at a dose of about 400 mg.

In some embodiments of the pharmaceutical composition, Compound 1 is administered at a maximum dose per day or per dose. In some embodiments, a total combined dose of 1g of Compound 1, or a pharmaceutically acceptable salt, solvate or polymorph thereof, is administered per day or per dose. In some embodiments, a total combined dose of 600 mg Compound 1, or a pharmaceutically acceptable salt, solvate or polymorph thereof, is administered per day or per dose. In some embodiments, a total combined dose of 500 mg Compound 1, or a pharmaceutically acceptable salt, solvate or polymorph thereof, is administered per day or per dose. In some embodiments, a total combined dose of 400 mg Compound 1, or a pharmaceutically acceptable salt, solvate or polymorph thereof, is administered per day or per dose. In some embodiments, a total combined dose of 300 mg Compound 1, or a pharmaceutically acceptable salt, solvate or polymorph thereof, is administered per day or per dose. In some embodiments, a total combined dose of 200 mg Compound 1, or a pharmaceutically acceptable salt, solvate or polymorph thereof, is administered per day or per dose. In some embodiments, Compound 1 or a pharmaceutically acceptable salt or polymorph thereof, is administered to a patient, wherein Compound 1 or a pharmaceutically acceptable salt or polymorph thereof is administered once a day. In some embodiments, the pharmaceutical composition is administered twice a day.

In some embodiments, Compound 1 or a pharmaceutically acceptable salt or polymorph thereof is administered to a patient, wherein Compound 1 or a pharmaceutically acceptable salt or polymorph thereof is administered once a day with food. It has been found that food can dramatically reduce the adverse event profile. The incidence and severity of the side effects, such as nausea, emesis and headache, can be reduced when Compound 1 or a pharmaceutically acceptable salt or polymorph thereof, is taken with food.

In some embodiments, Compound 1 or a pharmaceutically acceptable salt or polymorph thereof, is administered to a patient, wherein Compound 1 or a pharmaceutically acceptable salt or polymorph thereof is administered once a day for at least 7 days, 10 days, 2 weeks, 3 weeks, 4 weeks, 1 month, 2 months, 3 months, 4 months, 6 months, 7 months, 8 months, 9 months, 10 months, 11 months, a year, 1.5 years, or 2 years. In some embodiments, the patient is treated for 3 months. In some embodiments, the patient is treated for 6 months. In some embodiments, the patient is treated for 1 year. In some embodiments, the patient is treated for 1.5 years. In some embodiments, the patient is treated for 2 years, 3 years, 4 years, 5 years, over 5 years, or the duration of life.

In some embodiments, the pharmaceutical compositions are presented in a unit dosage form by methods known to those skilled in the art. For illustrative purposes, a typical unit dosage form for oral administration may contain from about 0.01 to about 1000 mg, from about 0.05 to about 500 mg, or from about 0.5 mg to about 200 mg.

### Combination Therapies

In one embodiment, the pharmaceutical composition comprising compounds of the present disclosure is used in combination with an additional active agent, such as Hydroxyurea (HU). The compounds of the present disclosure and the additional active agent may be administered simultaneously, sequentially, or at any order. The compounds of the present disclosure and the additional active agent may be administered at different dosages, with different dosing frequencies, or via different routes, whichever is suitable.

The term “administered simultaneously”, as used herein, is not specifically restricted and means that the compounds of the present disclosure and the additional active agent are substantially administered at the same time, e.g. as a mixture or in immediate subsequent sequence.

The term “administered sequentially”, as used herein, is not specifically restricted and means that the compounds of the present disclosure and the additional active agent are not administered at the same time but one after the other, or in groups, with a specific time interval between administrations. The time interval may be the same or different between the respective administrations of the compounds of the present disclosure and the additional active agent and may be selected, for example, from the range of 2 minutes to 96 hours, 1 to 7 days or one, two, or three weeks. Generally, the time interval between the administrations may be in the range of a few minutes to hours, such as in the range of 2 minutes to 72 hours, 30 minutes to 24 hours, or 1 to 12 hours. Further examples include time intervals in the range of 24 to 96 hours, 12 to 36 hours, 8 to 24 hours, and 6 to 12 hours.

The molar ratio of the compounds of the present disclosure and the additional active agent is not particularly restricted. For example, when the compounds of the present disclosure and one additional active agent are combined in a composition, the molar ratio of them may be in the range of 1:500 to 500:1, or of 1:100 to 100:1, or of 1:50 to 50:1, or of 1:20 to 20:1, or of 1:5 to 5:1, or 1:1. Similar molar ratios apply when the compounds of the present disclosure and two or more other active agents are combined in a composition. The compounds of the present disclosure compounds of the present disclosure may comprise a predetermined molar weight percentage from about 1% to 10%, or about 10% to about 20%, or about 20% to about 30%, or about 30% to 40%, or about 40% to 50%, or about 50% to 60%, or about 60% to 70%, or about 70% to 80%, or about 80% to 90%, or about 90% to 99% of the composition.

### Methods of Using Compounds of the Disclosure

PDE9 is expressed specifically in the human haematopoietic system including neutrophils, reticulocytes erythroid and erythroleukaemic cells. Furthermore, SCD patients exhibit a marked and significant elevation of PDE9 expression in reticulocytes and neutrophils compared to healthy individuals (Almeida et al., Br J Haematol. 2008 September; 142(5), 836). Evidence additionally demonstrates a link between PDE9 and cell adhesion since pharmacologic PDE9 inhibition ameliorates the increased adhesive properties of SCD neutrophils (Miguel et al., Inflamm Res. 2011 July; 60(7), 633). The mechanism by which PDE9 inhibition decreases cell adhesion has been shown to be mediated by increased cGMP and decreased endothelial adhesion molecule expression. Importantly, in an animal model of SCD, the PDE9 inhibitor-mediated decrease in cell adhesion had the functional effect of increased cell survival. In addition to demonstrating decreased cell adhesion comparable to HU, PDE9 inhibition resulted in increased fetal non-sickled haemoglobin (HbF) production, which reduced the cellular concentration of abnormal haemoglobin (HbS) within red blood cells (RBCs) resulting in less polymerization of the abnormal haemoglobin and its associated sequelae. The importance of increasing HbF in treating SCD is evidenced by results of large studies like the Cooperative Study of Sickle Cell Disease, as well as studies in a variety of patient cohorts outside of the United States, showing that HbF is among the most important modifiers of this disease (Alsultan et al., Am J Hematol. 2013, 88(6), 531) as well as data showing that modifiers of HbF improve other hematological parameters (Akinsheye, Blood, 2011, 118(1): 19). Finally, Almeida and colleagues demonstrated that treatment with HU combined with PDE9 inhibition in a mouse model of SCD leads to an additional beneficial amplification of the cGMP elevating effects of HU (Almeida et al., Blood. 2012 October; 120(14), 2879). In conclusion, PDE9 inhibition can modulate both the expression of fetal haemoglobin production as well as decrease cell adhesion, both mechanisms key for the treatment of SCD.

PDE9 inhibitors of the present disclosure and hydroxyurea (HU) act through different mechanisms. HU increases nitric oxide (NO) levels, which activate soluble guanylyl cyclase (sGC) to generate cGMP. PDE9 inhibitors of the present disclosure block the degradation of cGMP by inhibiting PDE9 enzymatic activity, thus elevating cGMP levels. In erythroid lineages, cGMP binds to protein kinase G (PKG) and signals synthesis of fetal gamma globin and ultimately production of HbF. In hematopoietic cells where PDE9 expression is high, the direct inhibition of PDE9 activity increases cGMP levels, which promotes decreased leucocyte adhesion.

One aspect of the present disclosure provides methods of using PDE9 inhibitors of the present disclosure and pharmaceutical compositions comprising PDE9 inhibitors of the present disclosure.

PDE9 inhibitors of the present disclosure may be used to treat sickle cell disease or any disease and/or symptom related to sickle cell disease, such as anemia, sickle-hemoglobin C disease (SC), vaso-occlusive crisis, attacks of pain (sickle cell crisis), splenic sequestration crisis, acute chest syndrome, aplastic crisis, hemolytic crisis, long-term pain, bacterial infections, and stroke.

In one embodiment, PDE9 inhibitors of the present disclosure are used to increase hemoglobin levels in the subject.

In another embodiment, PDE9 inhibitors of the present disclosure are used to increase cGMP levels in a cell or in the plasma of a subject, wherein the subject has sickle cell disease. The cell may be, but not limited to, red blood cells and/or white blood cells. The cGMP level may be increased by at least 50%, at least 100%, or at least 150%. In some embodiments, the cGMP level may be increased at least 2 times, 3 times, 4 times, 5 times, 10 times, 15 times, 20 times, or 25 times.

In another embodiment, PDE9 inhibitors of the present disclosure are used to increase fetal hemoglobin (HbF) positive red blood cell number in a subject, wherein the subject has sickle cell disease. The HbF positive red blood cell number is increased by at least 50%, at least 100%, or at least 150%. In some embodiments, the HbF positive red blood cell number is increased by at least 2 times, 3 times, 4 times, 5 times, 10 times, 15 times, 20 times, or 25 times.

In another embodiment, PDE9 inhibitors of the present disclosure are used to reduce sickle red blood cell percentage (% sickle RBC), stasis percentage (% stasis), total bilirubin, or total leucocyte count in a subject, wherein the subject has sickle cell disease. The % sickle RBC, % stasis, total bilirubin, total leucocyte count or spleen weight is decreased by at least 10%, 20%, 30%, 40%, 50%, 60% or 70%.

cGMP level may be measured with any suitable method in the art, such as enzyme immunoassay.

HbF positive cells, as used herein, means red blood cells with HbF. HbF positive cells may be measured from a blood sample with any suitable method in the art, such as electrophoresis and/or colorimetric methods.

Sickle red blood cells, sickled red blood cells, as used herein, means red blood cells with a crescent or sickle shape. % sickle red blood cell may be measured from a blood sample with any suitable method in the art.

Stasis or microvascular stasis, as used herein, is serious slowing, or complete cessation, of blood or lymph flow through vessels. % stasis is the number of static (no flow) venules divided by the number of flowing venules times 100. % stasis may be measured with any suitable method in the art.

Total bilirubin, as used herein, means both unconjugated and conjugated bilirubin. Total bilirubin levels may be measured from a blood sample with any suitable method in the art.

Total leucocyte count or total white blood cell count, as used herein, is a blood test that measures the number of white blood cells in the body. It may be measured from a blood sample with any suitable method in the art.

Another aspect of the present disclosure provides methods of using a PDE9 inhibitor of the present disclosure in combination with at least one other active agent. They may be administered simultaneously or sequentially. They may be present as a mixture for simultaneous administration, or may each be present in separate containers for sequential administration.

The term “simultaneous administration”, as used herein, is not specifically restricted and means that the PDE9 inhibitor of the present disclosure and the at least one other active agent are substantially administered at the same time, e.g. as a mixture or in immediate subsequent sequence.

The term “sequential administration”, as used herein, is not specifically restricted and means that the PDE9 inhibitor of the present disclosure and the at least one other active agent are not administered at the same time but one after the other, or in groups, with a specific time interval between administrations. The time interval may be the same or different between the respective administrations of PDE9 inhibitor of the present disclosure and the at least one other active agent and may be selected, for example, from the range of 2 minutes to 96 hours, 1 to 7 days or one, two or three weeks. Generally, the time interval between the administrations may be in the range of a few minutes to hours, such as in the range of 2 minutes to 72 hours, 30 minutes to 24 hours, or 1 to 12 hours. Further examples include time intervals in the range of 24 to 96 hours, 12 to 36 hours, 8 to 24 hours, and 6 to 12 hours.

The molar ratio of the PDE9 inhibitor of the present disclosure and the at least one other active agent is not particularly restricted. For example, when a PDE9 inhibitor of the present disclosure and one other active agent are combined in a composition, the molar ratio of them may be in the range of 1:500 to 500:1, or of 1:100 to 100:1, or of 1:50 to 50:1, or of 1:20 to 20:1, or of 1:5 to 5:1, or 1:1. Similar molar ratios apply when a PDE9 inhibitor of the present disclosure and two or more other active agents are combined in a composition. The PDE9 inhibitor of the present disclosure may comprise a predetermined molar weight percentage from about 1% to 10%, or about 10% to about 20%, or about 20% to about 30%, or about 30% to 40%, or about 40% to 50%, or about 50% to 60%, or about 60% to 70%, or about 70% to 80%, or about 80% to 90%, or about 90% to 99% of the composition.

The other active agent may be a different PDE9 inhibitor of the present disclosure or HU. The other active agent may also be an antibiotic agent such as penicillin, a nonsteroidal anti-inflammatory drug (NSAIDS) such as diclofenac or naproxen, a pain relief medication such as opioid, or folic acid.

Yet another aspect of the present disclosure provides methods of using a PDE9 inhibitor of the present disclosure in combination with at least one other therapy, such as but not limited to blood transfusion, bone marrow transplant, or gene therapy.

### Kits and Devices

The disclosure provides a variety of kits and devices for conveniently and/or effectively carrying out methods of the present disclosure. Typically, kits will comprise sufficient amounts and/or numbers of components to allow a user to perform multiple treatments of a subject(s) and/or to perform multiple experiments.

In one embodiment, the present disclosure provides kits for treating sickle cell disease, comprising a PDE9 inhibitor compound of the present disclosure or a combination of PDE9 inhibitor compounds of the present disclosure, optionally in combination with any other active agents, such as HU, an antibiotic agent such as penicillin, a nonsteroidal anti-inflammatory drug (NSAIDS) such as diclofenac or naproxen, a pain relief medication such as opioid, or folic acid.

The kit may further comprise packaging and instructions and/or a delivery agent to form a formulation composition. The delivery agent may comprise a saline, a buffered solution, or any delivery agent disclosed herein. The amount of each component may be varied to enable consistent, reproducible higher concentration saline or simple buffer formulations. The components may also be varied in order to increase the stability of PDE9 inhibitor compounds in the buffer solution over a period of time and/or under a variety of conditions.

The present disclosure provides for devices that may incorporate PDE9 inhibitor compounds of the present disclosure. These devices contain in a stable pharmaceutical formulation available to be immediately delivered to a subject in need thereof, such as a human patient with sickle cell disease.

Non-limiting examples of the devices include a pump, a catheter, a needle, a transdermal patch, a pressurized olfactory delivery device, iontophoresis devices, multi-layered microfluidic devices. The devices may be employed to deliver PDE9 inhibitor compounds of the present disclosure according to single, multi-or split-dosing regiments. The devices may be employed to deliver PDE9 inhibitor compounds of the present disclosure across biological tissue, intradermal, subcutaneously, or intramuscularly. More examples of devices suitable for delivering PDE9 inhibitor compounds include but not limited to a medical device for intravesical drug delivery disclosed in International Publication WO 2014036555, a glass bottle made of type I glass disclosed in US Publication No. 20080108697, a drug-eluting device comprising a film made of a degradable polymer and an active agent as disclosed in US Publication No. 20140308336, an infusion device having an injection micro-pump, or a container containing a pharmaceutically stable preparation of an active agent as disclosed in U.S. Pat. No. 5,716,988, an implantable device comprising a reservoir and a channeled member in fluid communication with the reservoir as disclosed in International Publication WO 2015023557, a hollow-fiber-based biocompatible drug delivery device with one or more layers as disclosed in US Publication No. 20090220612, an implantable device for drug delivery including an elongated, flexible device having a housing defining a reservoir that contains a drug in solid or semi-solid form as disclosed in International Publication WO 2013170069, a bioresorbable implant device disclosed in U.S. Pat. No. 7,326,421, contents of each of which are incorporated herein by reference in their entirety.

### Definitions

The articles “a” and “an,” as used herein, should be understood to mean “at least one,” unless clearly indicated to the contrary.

The phrase “and/or,” as used herein, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements).

As used herein, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements.

In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein, the phrase “at least one” in reference to a list of one or more elements should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, all transitional phrases such as “comprising,” “including,” “carrying,” “having,” “containing,” “involving,” “holding,” and the like are to be understood to be open-ended, i.e., to mean including but not limited to.

Only the transitional phrases “consisting of” and “consisting essentially of” shall be closed or semi-closed transitional phrases, respectively, as set forth in the United States Patent Office Manual of Patent Examining Procedures.

As used herein, a “subject” or a “patient” refers to any mammal (e.g., a human), such as a mammal that may be susceptible to a disease or disorder, for example, tumorigenesis or cancer. Examples include a human, a non-human primate, a cow, a horse, a pig, a sheep, a goat, a dog, a cat, or a rodent such as a mouse, a rat, a hamster, or a guinea pig. In various embodiments, a subject refers to one that has been or will be the object of treatment, observation, or experiment. For example, a subject can be a subject diagnosed with cancer or otherwise known to have cancer or one selected for treatment, observation, or experiment on the basis of a known cancer in the subject.

As used herein, “treatment” or “treating” refers to amelioration of a disease or disorder, or at least one sign or symptom thereof. “Treatment” or “treating” can refer to reducing the progression of a disease or disorder, as determined by, e.g., stabilization of at least one sign or symptom or a reduction in the rate of progression as determined by a reduction in the rate of progression of at least one sign or symptom. In another embodiment, “treatment” or “treating” refers to delaying the onset of a disease or disorder.

As used herein, “prevention” or “preventing” refers to a reduction of the risk of acquiring or having a sign or symptom a given disease or disorder, i.e., prophylactic treatment.

The phrase “therapeutically effective amount” as used herein means that amount of a compound, material, or composition comprising a compound of the present teachings that is effective for producing a desired therapeutic effect. Accordingly, a therapeutically effective amount treats or prevents a disease or a disorder, e.g., ameliorates at least one sign or symptom of the disorder. In various embodiments, the disease or disorder is a cancer.

A dash (“-”) that is not between two letters or symbols is used to indicate a point of attachment for a substituent. For example, −CONH₂ is attached through the carbon atom (C).

By “optional” or “optionally,” it is meant that the subsequently described event or circumstance may or may not occur, and that the description includes instances where the event or circumstance occurs and instances in which it does not. For example, “optionally substituted aryl” encompasses both “aryl” and “substituted aryl” as defined herein. It will be understood by those ordinarily skilled in the art, with respect to any group containing one or more substituents, that such groups are not intended to introduce any substitution or substitution patterns that are sterically impractical, synthetically non-feasible, and/or inherently unstable.

All numerical ranges herein include all numerical values and ranges of all numerical values within the recited range of numerical values. As a non-limiting example, (C₁-C₆) alkyls also include any one of C₁, C₂, C₃, C₄, C₅, C₆, (C₁-C₂), (C₁-C₃), (C₁-C₄), (C₁-C₅), (C₂-C₃), (C₂-C₄), (C₂-C₅), (C₂-C₆), (C₃-C₄), (C₃-C₅), (C₃-C₆), (C₄-C₅), (C₄-C₆), and (C₅-C₆) alkyls.

Further, while the numerical ranges and parameters setting forth the broad scope of the disclosure are approximations as discussed above, the numerical values set forth in the Examples section are reported as precisely as possible. It should be understood, however, that such numerical values inherently contain certain errors resulting from the measurement equipment and/or measurement technique.

### LIST OF ABBREVIATIONS AND TERMS

¹H-NMR: Proton Nuclear Magnetic Resonance spectroscopy
ADME: Absorption, Distribution, Metabolism, and Excretion
AE: Adverse event
AUC₀₋₂₄: area under the concentration-time curve from time 0 to 24 hours postdose
BBB: blood-brain barrier
Cₘₐₓ: maximum plasma concentration
cGMP: cyclic guanosine monophosphate
DMSO: dimethyl sulfoxide
DSFC: dorsal skin-fold chambers
F cells: blood cells with fetal haemoglobin
FIH: first in human
FTIR: Fourier transform infrared spectroscopy
GC: gas chromatography
HBB: hemoglobin subunit beta
HbF: fetal hemoglobin
HBG: gamma-globin gene
HbS: sickle hemoglobin
hERG: human ether-à-go-go related gene
HPLC: high-performance liquid chromatography
HU: hydroxyurea
IC: inhibitory concentration
IC₅₀: a half minimal inhibitory concentration
ICAM-1: intercellular adhesion molecule-1
ICH: International Conference on Harmonization
ICP-MS: inductively coupled plasma mass spectroscopy
IV: intravenous
MAD: multiple-ascending dose
MTD: maximum tolerated dose
NO: nitric oxide
NOAEL: no-observed-adverse-effect level
PD: pharmacodynamic
PDE9: phosphodiester-9
PEG polyethylene glycol
PIC: Powder in capsule
PK: pharmacokinetic(s)
PKG: protein kinase G
RBC: red blood cell
RH: relative humidity
SCD: sickle cell disease
SD: standard deviation
SEM: standard error of the mean
sGC: soluble guanylyl cyclase
t½: half-life
TK: Toxicokinetic
Tₘₐₓ: time of maximum concentration
VOC: vaso-occlusive crisis
WBC: white blood cell
w/w %: weight/weight percent

### EXAMPLES

It will be appreciated that the following examples are intended to illustrate but not to limit the present disclosure. Various other examples and modifications of the foregoing description and examples will be apparent to a person skilled in the art after reading the disclosure without departing from the spirit and scope of the disclosure, and it is intended that all such examples or modifications be included within the scope of the appended claims. All publications and patents referenced herein are hereby incorporated by reference in their entirety.

### Example 1. Synthesis and Formulation of Compound 1

Compound 1 is an enantiomer of 6-[4-methyl-1-(pyrimidin-2-ylmethyl)pyrrolidin-3-yl]-3-tetrahydropyran-4-yl-7H-imidazo[1,5-a]pyrazin-8-one disclosed in WO 2013/053690. Compound 1 may be prepared from chiral-selective purification from 6-[4-methyl-1-(pyrimidin-2-ylmethyl)pyrrolidin-3-yl]-3-tetrahydropyran-4-yl-7H-imidazo[1,5-a]pyrazin-8-one prepared according to the method disclosed in WO 2013/053690, the contents of which are incorporated herein by reference in their entirety. Compound 1 may also be prepared with the method disclosed in WO 2017/005786, the contents of which are incorporated herein by reference in their entirety.

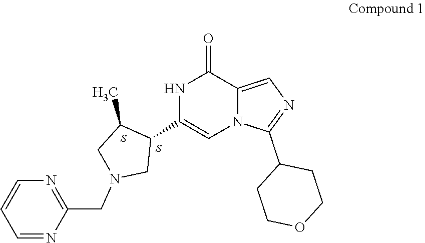

Compound 1 drug product to be used in ongoing clinical development is an immediate release tablet. The coating is may be used to assure uniformity of appearance across different tablet strengths and with the placebo.

Earlier clinical studies were performed with Compound 1 drug substance directly filled into opaque white gelatin capsules (Powder in Capsule, PIC) with no excipients or processing aids. An excipient-blended tablet form of the drug product for oral administration has been developed, as this allowed for scale-up of the manufacturing process and assurance of content uniformity. These tablets were tested for defined limits for purity, potency, dissolution, total aerobic microbial count, as well as total yeast and mold count. In addition, tests for specified microorganisms were performed.

Each tablet comprises 20 mg, 50 mg, 100 mg, or 200 mg of Compound 1 drug substance (the monohydrate of the API) or placebo. A representative tablet composition is shown below in Table 1.

TABLE 1
Compound 1 50 mg coated tablets

| Component | Weight/Unit (mg) |
|---|---|
| Tablet Blend | |
| Compound 1 Drug | 50.0 |
| Substance | |
| Microcrystalline | 318.0 |
| Cellulose | |
| Pre-gelatinized Starch | 20.0 |
| Colloidal Silicon Dioxide | 8.0 |
| Magnesium Stearate | 4.0 |
| Tablet Core Total | 400.0 |
| Coating Solution | |
| Opadry II White Film | 40.0 |
| Coating | |
| Purified Water | — |
| Final | 440.0 |

All tablets were configured such that the target weight of the core tablets was 400mg, and the target weight of the coated tablet was 440 mg. To accomplish this, the amounts of Compound 1 and Microcyrstalline Cellulose were adjusted accordingly. All other excipient amounts remained constant.

### Description of Manufacturing Process and Process Controls

The manufacture of the coated tablets follows common procedures for blending, tablet compaction, and coating.

### Roller Compaction of Drug Substance

Compound 1 drug substance was first processed by roller compaction to achieve a more uniform intermediate that is suitable for further blending and processing. A defined amount of API was passed through a roller compactor at 200-300 PSI and a roller speed of 4.0 RPM. The roller compacted material was passed through a 20-mesh screen to obtain a uniform particle size. The compacted and screened API was stored in double lined polyethylene bags pending use in tablet manufacturing.

### Blending of Roller Compacted Drug Substance with Excipients

A defined amount of the roller compacted API was mixed with the defined excipients (excluding the coating solution) using a 16 qt. V-shell Blender. Blending was performed in a manner such that not less than 300 revolutions of the blender were completed in the defined period. Samples were taken from multiple locations in the V-Shell Blender to verify the blend uniformity.

### Blended material was stored in a suitable HDPE container within double lined polyethylene bags pending further processing.

### Tableting

Tablet manufacturing was performed in a gravity fed tablet press with 11 mm upper and lower plain-faced tablet punches. The tablet press was adjusted to meet pre-determined specifications for tablet weight, hardness, thickness, friability, and disintegration, as defined below. All core tablets are screened through a metal detector and de-dusted prior to film coating.

### Film Coating:

A uniform coating mixture was prepared by mixing the defined amount of Opadry II White in Purified Water and mixing the suspension for no less than 45 minutes. Tablets were loaded on to the pan coating equipment and preheated to 43° C. prior to initiation of the coating operations. The preheated tablets were then coated in a 15-inch coating pan rotating at 10-15 RPM. The spray rate of the coating suspension was controlled at 10 grams per minute, and inlet air temperature maintained between 40-60° C.

Tablets were sampled at defined intervals and weighed to determine if the desired target weight gain (10%) has been met. Once coating is completed the coated tablets were allowed to cool to ≤30° C.

### Bulk Packaging

Coated tablets were sampled for release testing and then transferred into a HDPE container double lined with double lined polyethylene bags.

### Clinical Packaging

Packaging of tablets for clinical trials was performed, including inkjet labeling of each container with the batch number to ensure identification. Thirty-three (33) tablets were filled into each 60 cc round white HDPE bottle by manual count. The count was verified by a second person before each bottle was filled. Following filling each bottle was manually closed with a 33-mm white polypropylene child resistant cap, and induction sealed.

### Example 2. Development of 300 mg Tablet

Development activities for the 300 mg tablet have shown that the addition of Hydroxypropyl Cellulose (HPC-SSL-SFP) and elevation of Microcrystalline Cellulose (Avicel PH200) percent level resulted in acceptable results of an increased tablet hardness and decreased loss during friability testing. However, the addition of HPC has increased the mean disintegration time from less than one minute to approximately 37 minutes. Multiple percent levels of HPC-SSL-SPF were evaluated in the formulation for the 300 mg tablet. The formulations being evaluated are shown below in Table 2.

TABLE 2
Optimization of HPC-SSL-SPF Percent Levels Utilized in Formulation.

| | mg/unit | | | | |
|---|---|---|---|---|---|
| | Formulation | Formulation | Formulation | Formulation | Formulation |
| | A | B | C | D | E |
| Component | 0.5% HPC | 1.0% HPC | 1.5% HPC | 2.0% HPC | 2.5% HPC |
| Compound 1 | 300.0 | 300.0 | 300.0 | 300.0 | 300.0 |
| Drug Substance, | | | | | |
| Densified | | | | | |
| Microcrystalline | 84.30 | 82.20 | 80.10 | 78.00 | 75.90 |
| Cellulose NF/EP | | | | | |
| (Avicel PH200) | | | | | |
| Pre-Gelatinized | 21.00 | 21.00 | 21.00 | 21.00 | 21.00 |
| Starch NF/EP | | | | | |
| (Starch 1500) | | | | | |
| Hydroxypropyl | 2.10 | 4.20 | 6.30 | 8.40 | 10.50 |
| Cellulose (HPC- | | | | | |
| SSL-SFP) | | | | | |
| Colloidal Silicon | 8.40 | 8.40 | 8.40 | 8.40 | 8.40 |
| Dioxide, NF/EP | | | | | |
| (Cab-O-Sil) | | | | | |
| Magnesium | 4.20 | 4.20 | 4.20 | 4.20 | 4.20 |
| Stearate, NF/EP | | | | | |
| (non-bovine | | | | | |
| Hyqual) | | | | | |
| Total Weight | 420.0 | 420.0 | 420.0 | 420.0 | 420.0 |

Multiple formulations of different percent levels of Hydroxypropyl Cellulose (HPC-SSL-SFP) were evaluated to determine the formulation that yielded acceptable results. The formulation utilized for the development of the 300 mg film-coated tablets, is shown below in Table 3.

TABLE 3
Formulation for 300 mg Film-Coated Tablets

| | Percent | Amount per |
|---|---|---|
| Ingredient | Weight/Weight | Tablet (mg) |
| Compound 1 Drug Substance | 71.4 | 300.0 |
| Densified | | |
| Microcrystalline Cellulose, | 20.1 | 84.3 |
| NF/EP (Avicel PH 200) | | |
| Hydroxypropyl Cellulose (HPC- | 0.5 | 2.1 |
| SSL-SFP) | | |
| Pre-gelatinized starch NF/EP | 5.0 | 21.0 |
| (Starch 1500) | | |
| Colloidal Silicon Dioxide, | 2.0 | 8.4 |
| NF/EP (Cab-O-Sil) | | |
| Magnesium Stearate, NF/EP | 1.0 | 4.2 |
| (non-bovine Hyqual) | | |
| Total Core Tablet | 100.0 | 420.0 |
| Film-Coating | | |
| Opadry II, 85F18422, White | 4.8 | 20.0 |
| Total Coated Tablet | 104.8 | 440.0 |

### Blending/Granulation

The excipients for development were record for a batch size of 2,380 tablets. Compound 1 drug substance was roller compacted. Compound 1 and all excipients were screened through a 20-mesh hand screen in the following order: 1) Compound 1 Drug Substance; 2) Pre-gelatinized Starch (Starch 1500); 3) Colloidal Silicon Dioxide (Cab-O-Sil); and 4) Microcrystalline Cellulose (Avicel PH200).

Once the target weight was achieved, a hardness profile (tablet hardness vs. disintegration time) was established and samples were collected. Each sample was used to execute tablet physical testing to include weight, hardness, thickness, disintegration, and friability.

### In-Process Guidelines

Tablet Thickness Range: 4.65-4.85 mm.
Tablet Hardness Target: 10 kP.
Friability (USP Friability): no more than 0.3%, no capping.
Disintegration (per USP): All tablets completely disintegrate in no more than 15 minutes.

### Coating

Tablets from development batch was coated at target conditions to evaluate the core tablet ability to withstand pan coating. Prior to charging the tablets into the coating pan, the pan was sprayed with the Opadry II, 85F18422, White and was allowed to dry. Operators monitored the spraying and drying process to ensure no flaking of the coating occurred. This created a thin layer of coating to prevent tablet defects caused by the coating pan such as scuffing. The tablets were coated to a 4.8% weight gain with Opadry II, 85F18422, White.

### Results and Discussion

The goal of the development activities was to develop a formulation for the Compound 1 tablet which contained an optimal percent level of Hydroxypropyl Cellulose (HPC-SSL-SFP), yielding acceptable results. The formulation utilized was to produce a tablet with acceptable hardness, thickness, friability, and disintegration results. The tablets of each formulation were tested for hardness, thickness, friability, and disintegration. Target Results: (1) Thickness: 4.50-4.80 mm, (2) Friability: ≤0.3% loss, (3) Disintegration: ≤15 minutes, and (4) Hardness of about 10 kPa. Friability, disintegration and hardness were tested to USP

Compounding standards (United States Pharmacopeia and National Formulary are recognized standards in testing).

### Feasibility Batch With 0.5% HPC

The first formulation evaluated contained 0.5% Hydroxypropyl Cellulose (HPC-SSL-SFP), this formulation is shown below in Table 4.

TABLE 4
Formulation of Compound 1 300 mg tablets (0.5% HPC)

| | Weight per |
|---|---|
| Ingredient | Tablet (mg) |
| Compound 1 Drug | 300.0 |
| Substance, Densified | |
| Microcrystalline | 84.3 |
| Cellulose (Avicel PH | |
| 200) | |
| Hydroxypropyl | 2.1 |
| Cellulose (HPC-SSL- | |
| SFP) | |
| Pre-gelatinized Starch | 21.0 |
| (Starch 1500) | |
| Colloidal Silicon | 8.4 |
| Dioxide (Cab-O-Sil) | |
| Magnesium Stearate | 4.2 |
| (non-bovine Hyqual) | |
| Total Weight | 420.0 |

The tablets were to be prepared with a target fill weight of 420.0 mg, a thickness range of 4.50 mm-4.80 mm.

In order to test disintegration time, the tablets were submerged in water continuously until 6 tablets were completely disintegrated, the results are shown below in Table 5.

TABLE 5
Disintegration Time

| | | Compression |
|---|---|---|
| | Disintegration | Pressure |
| Tablet | Time (mm:ss) | (psi) |
| 1 | 03:24 | 2000 |
| 2 | 03:57 | 2000 |
| 3 | 06:40 | 2000 |
| 4 | 09:21 | 2600 |
| 5 | 12:40 | 2600 |
| 6 | 12:40 | 2600 |

The goal was to have all tablets completely disintegrate in 15 minutes or less. According to the data in Table 5 all tablets completely disintegrated in less than 15 minutes, meeting the goal time. The remaining tablet blend was compressed, using the target fill weight of 420.0 mg.

Another physical test performed was friability. The friability examines the tablets tendency for a tablet to chip, crumble or break following compression. The acceptance criteria for the batchs was a percent loss of 0.3% or less. The data collected during friability is shown below in Table 6.

TABLE 6
Friability

| Weight | | | | |
|---|---|---|---|---|
| Before | Weight | | % | # of |
| (g) | After(g) | Loss | Loss | Caps |
| 6.7378 | 6.7208 | 0.017 | 0.25 | 0 |

The 0.5% Hydroxypropyl Cellulose (HPC-SSL-SFP) percent level yielded results meeting the acceptance criterions provided in the batch record.

### Feasibility Batch With 1.5% HPC

A tablet using the formulation containing 1.5% Hydroxypropyl Cellulose (HPC-SSL-SFP) was compressed, physical testing was conducted on these tablets to see if they too yielded acceptable results. The formulation is shown below in Table 7.

TABLE 7
Formulation of Compound 1, 300 mg tablets (1.5% HPC)

| | Weight per Tablet |
|---|---|
| Ingredient | (mg) |
| Compound 1 Drug Substance, | 300.0 |
| Densified | |
| Microcrystalline Cellulose (Avicel | 80.10 |
| PH 200) | |
| Hydroxypropyl Cellulose (HPC- | 6.30 |
| SSL-SFP) | |
| Pre-gelatinized Starch (Starch 1500) | 21.0 |
| Colloidal Silicon Dioxide (Cab-O- | 8.4 |
| Sil) | |
| Magnesium Stearate (non-bovine | 4.2 |
| Hyqual) | |
| Total Weight | 420.0 |

The tablets were submerged in water continuously until 3 tablets were completely disintegrated. The results are shown in Table 8.

TABLE 8
Disintegration Time

| | Disintegration |
|---|---|
| Tablet | Time (mm:ss) |
| 1 | 15:19 |
| 2 | 15:28 |
| 3 | 16:57 |

The disintegration times all exceeded the acceptance criteria of 15 minutes indicating failing results for disintegration. The increased disintegration time is caused by the increased Hydroxypropyl Cellulose (HPC-SSL-SPF) level. It can be noted that the loss after friability is less than 0.3%.

After physical testing concluded, and the tablets were film coated to a weight gain of approximately 4.8% w/w. After comparing the results from the physical tests conducted on each of the formulations, it was determined that the Compound 1, 300 mg tablet (0.5% HPC) yielded the acceptable results for the tablet.

### Example 3. Compound 1 Reduces White Cell Adhesion and Activation

Polymorphic mononuclear cells (PMN), particularly neutrophils, play a critical role in pathogenesis of sickle cell disease (SCD) and activated neutrophils have been shown to be more adhesive to each other, platelets and the vascular endothelium. Recently several drugs targeting white cell binding to endothelial cells, have been advancing in clinical studies in patients. Compound 1 is able to increase expression of fetal hemoglobin in patient derived cells and murine models of SCD and reduce vessel occlusion in SCD murine models. In this Example, the ability of Compound 1 to reduce the adhesive properties of neutrophils from SCD patients and reduce sE-Selectin (sE-Sel) and markers of PMN activation in murine SCD models was studied.

Endothelial E-selectin (E-Sel) slows leukocyte rolling, which is followed by stationary adhesion and transmigration of activated leukocytes. Plasma levels of sE-Sel, produced by the enzymatic cleavage of the extracellular domains of E-Sel, are increased in SCD patients and this may be mediated by its interaction with leukocytes. In the Townes mouse model, plasma sE-Sel is increased 144% (139 mg/ml) over levels seen in control mice (57 mg/ml). This was reduced significantly in Townes mice treated with Compound 1, where plasma sE-Sel levels were elevated by only 61% over control mice (92 mg/ml).

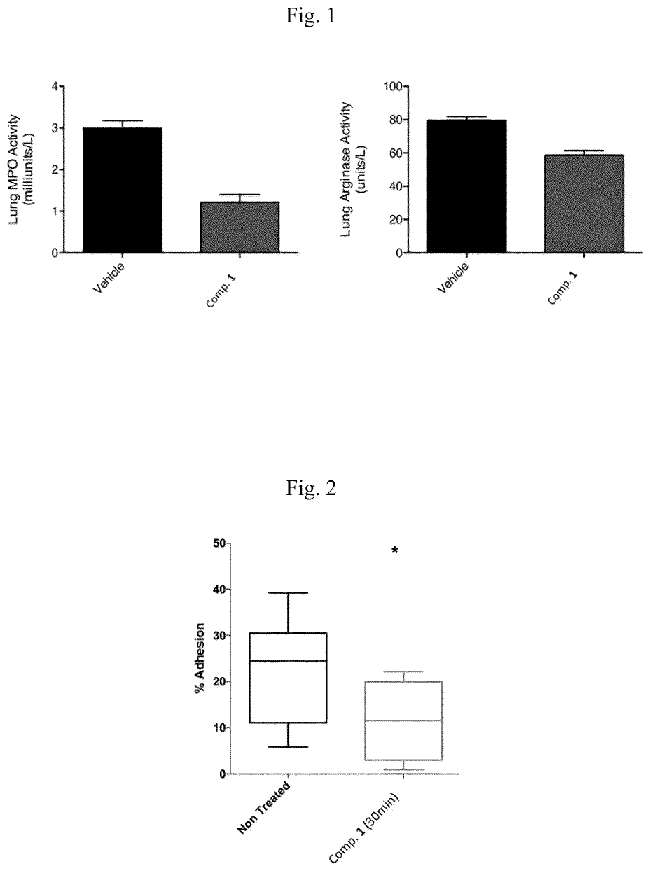

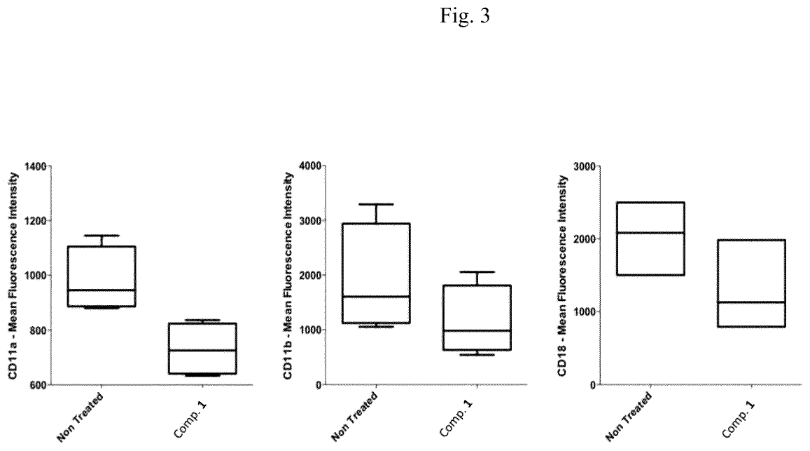

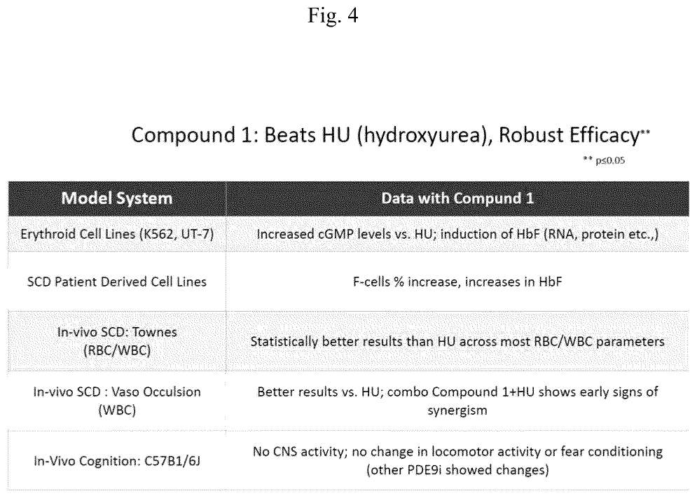

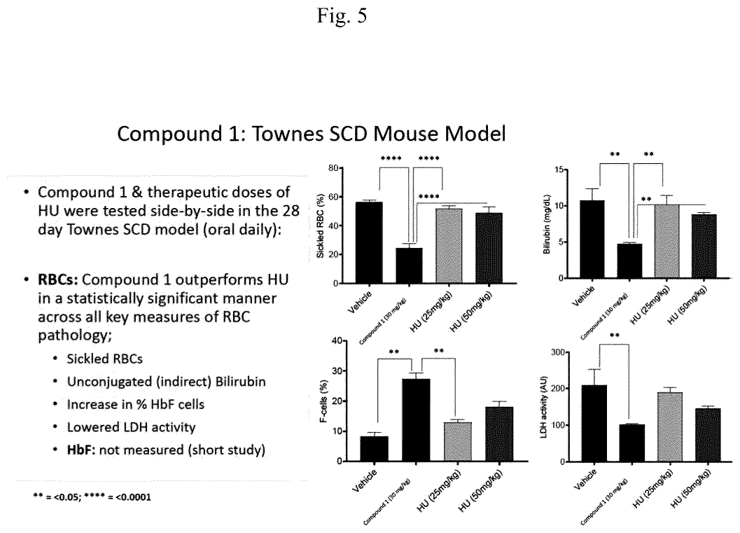

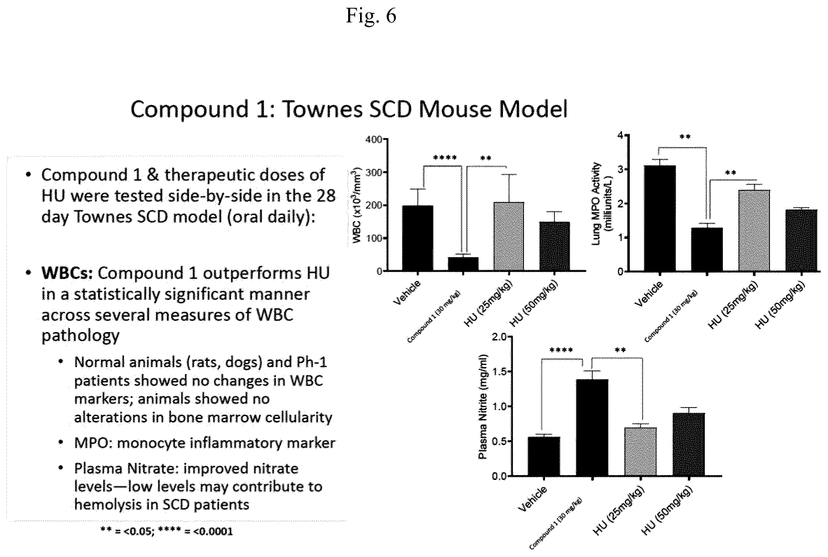

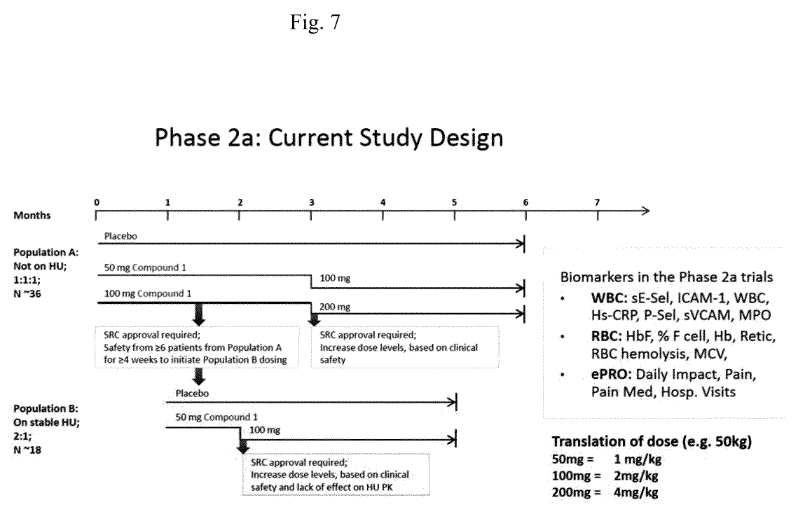

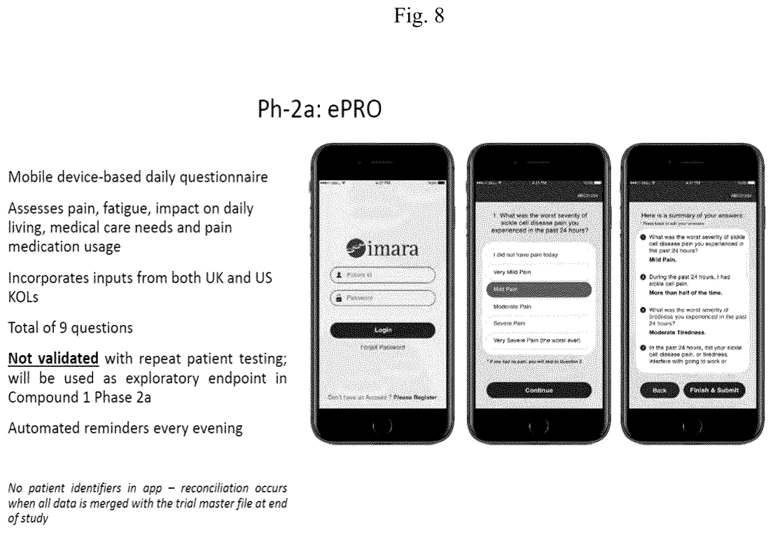

It was found that Compound 1 reduced circulating levels of PMNs in SCD models, but not in long term studies in healthy animals. This appears to be accompanied by a Compound 1-mediated reduction in disease specific cell activation including 67% lower levels of myeloid derived myeloperoxidase (MPO) and 26% lower levels of neutrophil derived arginase in the lung (FIG. 1). Using a previously described in vitro adhesion assay mimicking blood flow, where activated endothelial cells HMEC-1 line the inner surface of microchannels, perfused whole blood samples from SS (the most common form of sickle cell) patients showed that neutrophils aggregate and bind to the endothelial monolayer. This was quantified by real time monitoring of the green florescent patches in the mircochannel, as neutrophils are labeled by a specific Alexa Fluor® 488-conjugated antibody before the perfusion step. Untreated, patient neutrophils showed a significant amount of adhesion to activated HMEC-1. When added to blood samples prior to the perfusion step, Compound 1 reduced adhesions significantly and in a dose dependent manner. The inhibitory effect was initiated as early as 15 min of incubation, with the most potent inhibition of adhesion observed for 30 min incubation with 30 μM of Compound 1. Under these conditions, adhesion was reduced an average of 54% (p=0.03) (FIG. 2). Mechanistically, not willing to be bound by any theory, Compound 1 may target the stationary adhesion step of neutrophils as it lowered expression levels of key neutrophil integrins including CD11a [reduced 23% (p=0.002)], CD11b [reduced 39% (ns)) and CD18 [reduced 47% (p=0.03)) (FIG. 3).

Together, these data indicate a role for Compound 1 in reducing PBMCs mediated pathology in SCD by targeting the abnormal adhesion of neutrophils independently from their cell count in the circulation.

### Example 4. A Phase 2a, Randomised, Double-Blind, Placebo-Controlled Study of Compound 1 in Adult Patients with Sickle Cell Anaemia (SCA)

### Objectives:

Primary Objectives: To assess the safety and tolerability of Compound 1 in adult patients with sickle cell anemia (SCA), defined as homozygous sickle haemoglobin (HbSS) or sickle-β⁰ thalassemia, who are not receiving hydroxyurea (HU) and in adult SCA patients who are receiving a stable dose of HU.

Secondary Objectives: To characterize the pharmacokinetic (PK) profile of Compound 1 in adult patients with SCA who are/are not receiving a stable dose of HU; to characterize the PK profile of HU in adult patients with SCA before and after receiving Compound 1 to determine if there is a clinically relevant PK interaction.

Exploratory Objectives: To assess the pharmacodynamic (PD) effects of Compound 1 in adult patients with SCA who are/are not receiving stable HU; to assess the potential efficacy of Compound 1 on SCA-related clinical outcome measures in adult patients with SCA who are/are not receiving stable HU.

### Methodology

This is a randomized, double-blind, placebo-controlled study to evaluate the safety, tolerability, PK, and exploratory PD and clinical outcomes of the phosphodiesterase 9 (PDE9) inhibitor, Compound 1, administered once daily for 16 to 24 weeks in 2 populations of patients with SCA: those who are not receiving HU (Population A) and those who are currently receiving a stable dose of HU according to standard of care (Population B). Up to approximately 36 patients are enrolled in Population A and 18 patients are enrolled in Population B.
Population A: Following a Screening period of up to 4 weeks, eligible patients in Population A (i.e., those not receiving HU) receive either Compound 1 or placebo for a total of 24 weeks. On Day 1, patients are randomized 1:1:1 to receive oral Compound 1 30 mg, 50 mg, Compound 1 100 mg, or placebo daily for the first 12 weeks; for the second 12 weeks (Weeks 13-24), each patient's dose may be doubled (i.e., from 50 mg to 100 mg; from 100 mg to 200 mg; or placebo). (Note because placebo and all dose levels of Compound 1 are the same in appearance, dose escalation does not affect study medication blinding). Throughout the study, all available clinical data are reviewed approximately every 2 weeks, and dose escalation occurs on an individual patient basis on Day 85 only if approved based upon review of each patient's individual clinical safety data.
Population B: Following a Screening period of up to 4 weeks, eligible patients in Population B (i.e., those receiving stable HU) enter a lead-in period and have blood samples drawn to characterize the PK profile of the patient's prescribed dose of HU in the absence of Compound 1 (i.e., to characterize the patient's baseline HU PK profile). Two full baseline HU PK profiles (with blood samples drawn over a 10-hour period at least 48 hours apart) are determined.

Compound 1 dosing in Population B do not begin until at least 4 weeks of safety data from 6 patients in Population A have been reviewed and determined that it is safe and appropriate to begin dosing in Population B. Following approval to initiate dosing in Population B and once the baseline HU PK blood draws are complete, patients are randomized 2:1 on Day 1 to receive oral Compound 1 30 mg, 50 mg or placebo for 16 weeks. For the first 4 weeks (Weeks 1-4), patients receive study medication according to their randomized treatment assignment; for the following 12 weeks (Weeks 5-16), each patient's dose may be doubled (e.g., from 50 mg to 100 mg; or placebo). As in Population A, dose escalation occurs on Day 29 only if approved based upon review of each patient's individual clinical safety data.

### Study Design Rationale

This is the first study in a patient population (patients with SCA), and as such, is designed to examine the safety, tolerability, and PK, as well as the potential PD effects and clinical efficacy, of Compound 1 across a range of doses in adult patients with SCA. Given the possibility that Compound 1, if approved, could be administered as a single agent or co-administered with HU, the effects of Compound 1 are evaluated in SCA patients who are not receiving HU or any other treatment known to modulate HbF levels (Population A) as well as in those who are currently receiving a stable dose of HU (Population B).

Available nonclinical and healthy volunteer clinical data suggest that Compound 1 is safe and well tolerated at once daily doses of 30 50, 100, and 200 mg and that a potentially clinical beneficial PD effect is likely to be observed when a dose of at least 100 mg is administered for at least 24 weeks. Therefore, Population A is designed to explore the PD dose response in patients as well as the tolerability of the 200 mg dose level in sickle cell patients who have tolerated the 100 mg dose well.

Results from Population B are intended to provide information on Compound 1 when administered concomitantly with HU, both of which increase HbF levels through alternative biochemical pathways that increase intracellular cGMP. Because there are no clinical data to support administration of Compound 1 concomitantly with HU, patients in Population B initiate Compound 1 dosing at the low dose (30 mg or 50 mg) used in Population A and only escalate to the 100 mg dose if the 50 mg dose has been safe and tolerated for 4 weeks. In addition, although available nonclinical data do not suggest that concomitant administration of HU with Compound 1 would increase Compound 1 exposure, dosing in Population B does not initiate until 4 weeks of safety data are available from Population A in 2 patients each at 30 mg or 50 mg (starting dose in Population B) and at 100 mg (2× the starting dose) as well as placebo.

### Diagnosis and Main Criteria for Inclusion

### Inclusion Criteria:

Each patient must meet all of the following criteria to be enrolled in the study: 1. Male or female ≥18 or ≤50 years of age. 2. Confirmed diagnosis of SCA (HbSS or sickle-β0 thalassemia). Note, if not already documented in the patient's record, the diagnosis of SCA must be confirmed via electrophoresis, HPLC, and/or genotyping. 3. Use of HU: For patients in the Population A: Have not received HU within 90 days prior to Screening and are not planning to take HU within the next 6 months. For patients in Population B: Have received HU for at least 6 months, have been on a stable dose for at least 60 days prior to Screening, and are not planning to change the dose level, dose regimen, or discontinue HU within the next 6 months. 4. Female patients must not be pregnant and be highly unlikely to become pregnant. Male patients must be unlikely to impregnate a partner.

### Exclusion Criteria

Patients who meet any of the following criteria are excluded from the study: 1. Total Hb at Screening >11.0 g/dL or <6 g/dL. 2. Reticulocyte count <100×109/L. 3. >3 hospitalizations (for at least 24 hours) for vaso-occlusive crises (VOC), including acute chest syndrome (ACS) and priapism, within the prior year. 4. Receiving chronic outpatient opioid treatment (equivalent to >10 mg oral morphine daily) for any reason other than avascular necrosis (AVN). Note: chronic treatment is defined as continuous daily opioid use for ≥8 weeks. 5. Blood transfusion or donation of blood or any blood product within 60 days of Day 1 or on chronic transfusion therapy regimen. 6. Positive for human immunodeficiency virus (HIV), hepatitis C (HCV) antibodies (unless the patient has successfully completed drug therapy that results in cure/clearance of HCV), and hepatitis B surface antigen (HBsAg). 7. For female patients of childbearing potential, a positive serum human chorionic gonadotropin (hCG) test (Screening) or a positive urine hCG test on Day 1. 8. Estimated glomerular filtration rate (eGFR) <50 mL/min as calculated by the equation from the Modification of Diet in Renal Disease (MDRD) Study using creatinine, age, sex, and ethnicity. 9. Alanine aminotransferase (ALT) or aspartate aminotransferase (AST) >3× the upper limit of normal (ULN). 10. Body Mass Index (BMI) <17.5 or >35 kg/m2; a total body weight <50 kg. 11. Use of PDE5 inhibitors (including but not limited to sildenafil, tadalafil, vardenafil) within 7 days prior to the first dose of study drug, or planning to use any time during study. 12. A history of drug or alcohol abuse as judged by the investigator within the past 1 year, or a positive alcohol (breathalyzer) test (Screening or Day-1). 13. A cancer that has not been in complete remission for at least 5 years. Patients with squamous cell or basal cell carcinoma of the skin, localised cervical cancer, or localised prostate cancer are eligible if, in the opinion of the investigator, the condition has been adequately diagnosed, and is determined to be clinically in remission, and the patient's participation in the study would not represent a safety concern. 14. A history of a clinically significant allergic reaction or hypersensitivity, as judged by the investigator, to any drug or any component of the study drug formulations used in the study. 15. On ECG, a corrected QT interval, Fridericia's formula (QTcF) >450 ms in men and >470 ms in women or the presence of clinically significant abnormalities as determined by the investigator. 16. A history of major surgery within 4 weeks or minor surgery within 2 weeks of Day 1. 17. Any flu-like syndrome or other respiratory infection within 2 weeks of Day 1 or vaccination with attenuated live virus within 4 weeks of Day 1. 18. Participation in an investigational drug or device study within 30 days prior to Day 1. 19. Use within 30 days prior to Day 1, or planning to use during the study, of any drugs or substances that are known to strongly inhibit or induce cytochrome P450 enzymes (CYPs), including but not limited to cimetidine, cyclosporine, erythromycin, omeprazole, rifampin, ritonavir, and St. John's wort. If there is any question as to whether a substance is permitted, please review the product labelling (if applicable) and consult the Sponsor. 20. Consumption of grapefruit, grapefruit juice, or grapefruit products within 24 hours prior to Day 1 or planning to consume grapefruit products during the study. 21. Use within 30 days prior to Day 1, or planning to use during the study, of any CYP3A sensitive substrates, (excluding opioids), including but not limited to alfentanil, avanafil, budesonide, buspirone, conivaptan, darifenacin, darunavir, dasatinib, dronedarone, ebastine, eletriptan, eplerenone, everolimus, felodipine, ibrutinib, indinavir, lomitapide, lurasidone, maraviroc, midazolam, naloxegol, nisoldipine, quetiapine, saquinavir, sirolimus, tacrolimus, ticagrelor, tipranavir, tolvaptan, triazolam. 22. Use within 30 days prior to Day 1, or planning to use during the study, of any drugs or substances known to be significant substrates or inhibitors of P-glycoprotein (P-gp), including but not limited to cyclosporine, lovastatin, propranolol, quinidine, and simvastatin. If there is any question as to whether a substance is permitted, please review the product labelling (if applicable) and consult the Sponsor. 23. Other prior or ongoing medical condition, physical findings, or laboratory abnormality that, in the investigator's opinion, could adversely affect the safety of the patient, make it unlikely that the course of treatment or follow-up would be completed, or impair the assessment of study results.

### Investigational Product, Dosage and Mode of Administration

Compound 1 is supplied as 50, 100 or 200 mg white tablets and is administered orally with food. The different doses of Compound 1 are visually identical in tablet form.

### Reference Therapy, Dosage and Mode of Administration

Placebo consists of tablets containing matrix absent Compound 1 and is identical in appearance to the Compound 1 tablets. Placebo is administered orally with food.

### Duration of Treatment

The total duration of the study is approximately 32 weeks for Population A, including a Screening period of up to 4 weeks, a treatment period of 24 weeks, and a 4-week follow-up assessment after the last dose of study drug is administered.

The total duration of the study is approximately 32 weeks for Population B, including a Screening period of up to 4 weeks, a lead-in period of approximately 8 weeks, a treatment period of 16 weeks, and a 4-week follow-up assessment after the last dose of study drug is administered.

### Endpoints

The endpoints for Populations A and B are the same except where noted otherwise.
Primary Endpoints: Compound 1 safety and tolerability as measured by: Incidence and severity of adverse events (AEs) and serious adverse events (SAEs); Change from baseline in 12-lead electrocardiogram (ECG) parameters, clinical laboratory tests (chemistry, hematology, coagulation, urine), and vital signs; Physical examination findings.
Secondary Endpoints: The plasma PK profile of Compound 1 after oral administration to adult patients with SCA (Populations A and B); The plasma PK profile of HU before and after oral administration of Compound 1 to adult patients with SCA (Population B only).

Exploratory Endpoints: Compound 1 PD as measured by the following (additional exploratory biomarkers may also be tested): Total haemoglobin (Hb) levels; HbF value (%); % F cells; Indices of red cell haemolysis (unconjugated bilirubin, reticulocyte count, lactase dehydrogenase [LDH], and haptoglobin levels); Soluble E-selectin (sE-Sel), Soluble P-selectin (sP-Sel) and soluble intercellular adhesion molecule 1 (sICAM-1); High sensitivity-C reactive protein (hs-CRP). Compound 1 clinical outcomes as measured by pain-related measures (frequency, severity, and duration of pain; impact of pain/fatigue on work/school and on activities of daily living; need for/use of pain medication; SCA-related events requiring professional medical or health care, including events requiring hospitalization or therapies, such as transfusions) and in the physical, social, and emotional impact of SCA as measured by the Adult Sickle Cell Quality-of-Life Measurement Information System (ASCQ-Me).

In addition, a separate blood sample is collected for confirmation of diagnosis by electrophoresis, high performance liquid chromatography (HPLC) and/or DNA sequencing (as needed) as well as for possible pharmacogenomic analyses of genes that may affect treatment response (including but not limited to alpha globin and BCL11A).

What is claimed is:

1. An oral pharmaceutical composition comprising:
(i) about 100 mg to about 300 mg of 6-[(3S,4S)-4-methyl-1-(pyrimidin-2-ylmethyl)pyrrolidin-3-yl]-3-tetrahydropyran-4-yl-7H-imidazo[1,5-a]pyrazin-8-one (Compound 1), or a pharmaceutically acceptable salt, solvate, or polymorph thereof;
(ii) a filler selected from about 4% to about 6% by weight of pre-gelatinized starch and/or from about 15% to about 50% microcrystalline cellulose; and
(iii) a processing aid selected from about 1% to about 2.5% by weight of colloidal silicon dioxide and/or from about 0.5% to about 1.5% by weight of magnesium stearate, wherein the pharmaceutical composition is in the form of a solid tablet suitable for administration to a patient.

2. The pharmaceutical composition of claim 1, the composition having a friability of no more than about 0.3% weight loss and a having a disintegration time of less than about 15 minutes, as determined by USP friability and USP disintegration tests.

3. The pharmaceutical composition of claim 1, the composition having at least one of a friability of no more than about 0.3% weight loss or a disintegration time of less than about 15 minutes, as determined by USP friability and USP disintegration tests.

4. The pharmaceutical composition of any of the preceding claims, further comprising hydroxypropyl cellulose (HPC).

5. The pharmaceutical composition of claim 4, wherein the hydroxypropyl cellulose (HPC) is present in an amount from about 0.4% to about 0.5% by weight.

6. The pharmaceutical composition of claim 4, wherein the hydroxypropyl cellulose (HPC) is present in an amount of about 0.5% by weight.

7. The pharmaceutical composition of any one of claims 1 to 6, the composition having a hardness of about 10 kPa and/or a thickness of about 4.50 to about 4.80 mm.

8. The pharmaceutical composition of any one of claims 1 to 7, comprising about 100 mg, 200 mg, or about 300 mg of Compound 1, or a pharmaceutically acceptable salt, solvate, or polymorph thereof.

9. The pharmaceutical composition of claim 8, comprising about 300 mg of Compound 1, or a pharmaceutically acceptable salt, solvate, or polymorph thereof.

10. The pharmaceutical composition of any one of claims 1 to 9, comprising about 5% by weight of pre-gelatinized starch.

11. The pharmaceutical composition of any one of claims 1 to 10, comprising about 20% by weight of microcrystalline cellulose.

12. The pharmaceutical composition of any one of claims 1 to 11, comprising about 2% by weight of colloidal silicon dioxide.

13. The pharmaceutical composition of any one of claims 1 to 12, comprising about 1% by weight of magnesium stearate.

14. The pharmaceutical composition of any one of claims 1 to 13, the composition comprising pre-gelatinized starch, colloidal silicon dioxide, and magnesium stearate at a weight ratio of 5:2:1.

15. The pharmaceutical composition of any one of claims 1 to 14, further comprising a coating selected from an enteric coating or Opadry® II white film coating.

16. The pharmaceutical composition of claim 15, wherein the coating is about 2.5% by weight of the total table.

17. An oral pharmaceutical composition comprising:
from about 100 mg to about 300 mg of 6-[(3S,4S)-4-methyl-1-(pyrimidin-2-ylmethyl)pyrrolidin-3-yl]-3-tetrahydropyran-4-yl-7H-imidazo[1,5-a]pyrazin-8-one (Compound 1), or a pharmaceutically acceptable salt, solvate, or polymorph thereof;
about 5% by weight of pre-gelatinized starch;
about 20% by weight of microcrystalline cellulose;
about 2% by weight of colloidal silicon dioxide; and
about 1% by weight of magnesium stearate, wherein the pharmaceutical composition is in the form of a solid tablet.

18. The pharmaceutical composition of claim 17, the composition having a friability of no more than about 0.3% weight loss and/or a disintegration time of less than about 15 minutes, as determined by USP friability and USP disintegration tests.

19. The pharmaceutical composition of claim 17 or 18, further comprising about 0.5% by weight of hydroxypropyl cellulose (HPC).

20. An oral pharmaceutical composition comprising 6-[(3S,4S)-4-methyl-1-(pyrimidin-2-ylmethyl)pyrrolidin-3-yl]-3-tetrahydropyran-4-yl-7H-imidazo[1,5-a]pyrazin-8-one (Compound 1), or a pharmaceutically acceptable salt, solvate, or polymorph thereof; wherein the pharmaceutical composition is in the form of a solid tablet.

21. The pharmaceutical composition of claim 20, further comprising at least a filler.

22. The pharmaceutical composition of claim 21, wherein the filler is microcrystalline cellulose or pre-gelatinized starch.

23. The pharmaceutical composition of claim 22, the composition comprising about 4% to about 6% by weight of pre-gelatinized starch.

24. The pharmaceutical composition of claim 22, the composition comprising about 15 mg to about 25 mg of pre-gelatinized starch per tablet.

25. The pharmaceutical composition of any one of claims 20 to 24, further comprising at least a processing aid.

26. The pharmaceutical composition of claim 25, wherein the processing aid is colloidal silicon dioxide and/or magnesium stearate.

27. The pharmaceutical composition of claim 26, the composition comprising about 1% to about 2.5% by weight of colloidal silicon dioxide and/or about 0.5% to about 1.5% by weight of magnesium stearate.

28. The pharmaceutical composition of claim 26, the composition comprising about 6 mg to about 8 mg colloidal silicon dioxide and/or about 2 mg to about 4 mg of magnesium stearate per tablet.

29. The pharmaceutical composition of any one of claims 20 to 28, comprising microcrystalline cellulose, pre-gelatinized starch, colloidal silicon dioxide, and magnesium stearate.

30. The pharmaceutical composition of any one of claims 20 to 29, further comprising a coating selected from an enteric coating or Opadry® II white film coating.

31. The pharmaceutical composition of claim 30, wherein the coating is about 2.5% by weight of the tablet.

32. The pharmaceutical composition of claim 30, the composition comprising about 20 mg to about 40 mg of the coating per tablet.

33. The pharmaceutical composition of any one of claims 20 to 32, the composition comprising pre-gelatinized starch, colloidal silicon dioxide, and magnesium stearate at a weight ratio of 5:2:1.

34. The pharmaceutical composition of any one of claims 20 to 33, wherein Compound 1, or a pharmaceutically acceptable salt or polymorph thereof, is present in an amount of about 30 mg, 50 mg, 100 mg, 150 mg, 200 mg, 250 mg, 300 mg, or 350 mg.

35. The pharmaceutical composition of any one of claims 20 to 33, wherein Compound 1, or a pharmaceutically acceptable salt or polymorph thereof, is present in the composition in an amount from about 50% to about 80% or from about 60% to about 75% by weight of the solid tablet.

36. The pharmaceutical composition of claim 35, wherein Compound 1, or a pharmaceutically acceptable salt or polymorph thereof, is present in the composition in an amount 65%, about 68%, about 70%, about 72%, or about 75% by weight of the solid tablet.

37. The pharmaceutical composition of any one of claims 20 to 36, the composition having a friability and/or a disintegration time.

38. The pharmaceutical composition of claim 37, the composition having a friability of no more than 0.3% weight loss and/or a disintegration time of less than about 15 minutes, wherein friability and disintegration time are determined by USP testing.

39. The pharmaceutical composition of any one of claims 20 to 38, the composing further comprising 0.4% to about 0.5% by weight of hydroxypropyl cellulose.

40. A method for treating sickle cell disease in a subject in need, comprising administering the pharmaceutical composition of any one of claims 1 to 39.

41. The method of claim 40, wherein the pharmaceutical composition is taken with food.

42. The method of claim 40, wherein the pharmaceutical composition is administered once per day, twice per day, or three times per day.

43. The method of claim 40, wherein the pharmaceutical composition is administered once per day.

44. The method of any one of claims 40 to 43, wherein the pharmaceutical composition is administered for at least 4 weeks, 12 weeks, 16 weeks, or 24 weeks.

45. The method of any one of claims 40 to 44, further comprising administering hydroxyurea (HU).

46. The method of any one of claims 40 to 45, comprising administering to the subject about 0.3 mg/kg to about 6.0 mg/kg or from about 0.3 mg/kg to about 1.0 mg/kg of subject's body mass per day or per dose of Compound 1, or a pharmaceutically acceptable salt or polymorph thereof.
